(19) United States
(12) Patent Application Publication
(10) Pub. No.: US 2025/0179575 A1
(43) Pub. Date: Jun. 5, 2025

(54) Liquid Biopsy Analysis of Cellular States to Predict Immunotherapy Toxicity

(71) Applicants: Washington University, St. Louis, MO (US); The Board of Trustees of the Leland Stanford Junior University, Stanford, CA (US)

(72) Inventors: Aadel Chaudhuri, St. Louis, MO (US); Aaron M. Newman, Palo Alto, CA (US); Alexander Lozano, Stanford, CA (US)

(73) Assignees: Washington University, St. Louis, MO (US); The Board of Trustees of the Leland Stanford Junior University, Stanford, CA (US)

(21) Appl. No.: 18/729,150

(22) PCT Filed: Jan. 12, 2023
(86) PCT No.: PCT/US2023/060573

§ 371 Date: Jul. 15, 2024

Related U.S. Application Data
(60) Provisional application No. 63/299,377, filed on Jan. 13, 2022.

(51) Int. Cl. C12Q 1/6883; G01N 33/569; G01N 33/68

(52) U.S. Cl. CPC C12Q 1/6883; G01N 33/56972; G01N 33/6854; G01N 2333/7051; G01N 2800/52

(57) ABSTRACT

Methods are disclosed for predicting a likelihood of developing a severe immune-related adverse event (irAE) associated with the administration of an immunotherapy in a melanoma patient based on abundances of activated CD4 memory T cells and/or diversities of T cell receptors (TCR) within a peripheral blood sample obtained from the patient.

### CROSS-REFERENCE TO RELATED APPLICATIONS

This application claims priority from U.S. Provisional Application Ser. No. 63/299,377 filed on Jan. 13, 2022, which is incorporated herein by reference in its entirety.

### STATEMENT REGARDING FEDERALLY SPONSORED RESEARCH OR DEVELOPMENT

This invention was made with government support under CA187192 and CA238711 awarded by the National Institutes of Health. The government has certain rights in the invention.

### MATERIAL INCORPORATED-BY-REFERENCE

Not applicable.

### FIELD OF THE INVENTION

The present disclosure generally relates to methods for predicting immunotherapy toxicity in patients.

### BACKGROUND OF THE INVENTION

Although ICIs have revolutionized cancer treatment, approximately 10-60% of ICI-treated patients with melanoma currently develop severe immune-related toxicities, with the rate of toxicity closely linked to the specific therapy administered. Also known as irAEs, ICI-induced toxicities impact a range of organ systems, including the lungs, liver, heart, skin, pituitary gland, and gastrointestinal tract, and can be associated with substantial morbidity requiring urgent medical intervention. Such morbidities can lead to the suspension of anticancer treatment, and in the most severe cases, death. The biological drivers of irAEs are poorly characterized and there is no method in standard clinical practice to identify which patients are at the highest risk for developing them.

Accordingly, several groups have investigated potential biomarkers of ICI-induced toxicity based on blood or tumor analysis. However, these studies have generally been focused on early on-treatment prediction or single organ systems, with only modest performance for predicting irAEs in the pretreatment setting independent of the affected organ system. Recently, a candidate pneumonitis-only irAE biomarker using tumor immunohistochemistry was reported; however, this biomarker was indirectly identified from The Cancer Genome Atlas, which lacks toxicity annotations, and was evaluated in a case-control setting without the inclusion of low-grade irAEs. Another group identified a single-nucleotide polymorphism within the gene encoding microRNA-146a that was associated with severe irAE development. Still, other groups have identified ICI response biomarkers without examining irAEs.

Given the considerable heterogeneity of ICI-induced irAEs, including variation in their timing, severity, and location, determining the factors that cause them has remained challenging. Pre-existing autoantibodies, autoreactive tissue-resident T cells, and T cells with specificity for viral antigens stemming from chronic viral infection have all been implicated in irAEs. Changes in the gut microbiome leading to increased colonic interleukin-1β expression were also recently reported in ICI-induced colitis. Given these observations, several groups have investigated parallels between irAEs and autoimmune disease. Indeed, case reports have shown that ICIs can cause frank autoimmunity, suggesting that irAEs could represent subclinical autoimmunity in a subset of patients. However, whether a common immunological state precedes distinct manifestations of ICI-induced toxicity is unknown.

### SUMMARY OF THE INVENTION

Among the various aspects of the present disclosure is the provision of methods and compositions for the prediction of the likelihood of developing a severe immune-related adverse event (irAE) in a patient receiving immunotherapy based on a biomarker derived from a peripheral blood sample obtained from the patient prior to receiving the immunotherapy. In one aspect, disclosed methods include obtaining a peripheral blood sample from a subject prior to receiving an immunotherapy treatment and quantifying an abundance of activated CD4 memory T cells and a diversity of T cell receptors (TCR) in the peripheral blood sample. Preferred methods additionally include classifying the patient as likely to develop a severe irAR if the abundance of activated CD4 memory T cells in combination with the diversity of T cell receptors (TCR) exceeds a threshold (sometimes referred to herein as a model index). The threshold can be determined using a model index that identifies levels of activated CD4 memory T cells and TCR diversity and provides a range of values that represent a ceiling beyond which the patient is susceptible to irAR. In one aspect, a value of the model index (the combination of CD4 memory T cells and TCR diversity values) that exceeds a predetermined threshold is predictive of a more severe irAR. In an additional aspect, the method further includes determining the threshold value by reference to known clinical standards. In another aspect, the disclosed methods include determining the abundance of activated CD4 memory T cells and the diversity of T cell receptors (TCR) using at least one of: bulk RNA-sequencing (CIBERSORTx and MIXCR), mass cytometry by time of flight (CyTOF), immunoSEQ® TCR-β profiling, droplet-based scRNA-sequencing and scTCR-sequencing, and targeted RNA-sequencing using an RNA panel targeted to activated CD4 memory T cells.

In other aspects of the present disclosure, methods for predicting a likelihood of developing a severe immune-related adverse event (irAE) in a patient receiving an immunotherapy are disclosed. In one aspect, the methods include obtaining a first peripheral blood sample from a subject prior to receiving an immunotherapy treatment and a second peripheral blood sample subsequent to the administration of the immunotherapy. The disclosed methods in these other aspects include quantifying a first TCR diversity level from the first peripheral blood sample and a second TCR diversity level from the second peripheral blood sample. The disclosed methods further methods include obtaining a degree of TCR expansion by subtracting the first TCR diversity level from the second TCR diversity level. The disclosed methods further include classifying the patient as likely to develop severe irAR if the degree of TCR expansion exceeds a threshold value. In one aspect, the methods include predicting a time of onset of the severe irAR based on the degree of TCR expansion, wherein a higher degree of TCR expansion is predictive of an earlier onset of severe irAR. In one aspect, the methods include determining the diversity levels of T cell receptors (TCR) using at least one of: bulk RNA-sequencing (CIBERSORTx and MIXCR), mass cytometry by time of flight (CyTOF), immunoSEQ® TCR-β profiling, droplet-based scRNA-sequencing and scTCR-sequencing, and targeted RNA-sequencing using an RNA panel targeted to activated CD4 memory T cells.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a schematic of a study schema described in the present disclosure, including an overview of patients included in this study, a summary of their irAE status, exclusion criteria, and downstream analyses that were performed. Among 78 total eligible patients, 71 were evaluable for irAE analysis after exclusion criteria were applied.

FIG. 2A is a set of color-coded charts representing the characteristics of the single-cell discovery cohort from FIG. 1, including the highest irAE grade experienced and durable clinical response status after the start of immunotherapy.

FIG. 2B is a UMAP chart of a viSNE projection of peripheral blood cells analyzed by CyTOF. t-SNE, t-distributed stochastic neighbor embedding.

FIG. 2C is a (Left) heatmap showing the relative abundance of 20 cell states identified by CyTOF in 18 patients, grouped by future irAE status, as well as (Right) a graph showing the association of cell state abundance with severe irAE development. Statistical significance was determined by a two-sided, unpaired Wilcoxon rank-sum test and expressed as directional −log 10 P values. For associations with no severe irAE, −log 10 P values were multiplied by −1. Q values were determined by the Benjamini-Hochberg method.

FIG. 2D is a graph of the frequencies of CD4 TEM cells (CyTOF) in the pretreatment peripheral blood of patients stratified by future irAE status (no severe irAE, n=10 patients; severe irAE, n=8 patients). The box center lines, box bounds, and whiskers denote the medians, first and third quartiles, and minimum and maximum values, respectively. Statistical significance was determined by a two-sided, unpaired Wilcoxon rank-sum test.

FIG. 3A is a UMAP of peripheral blood cells profiled by scRNA-seq from 13 patients coanalyzed by CyTOF (FIG. 2A), colored by cell type, patient, and state (n=32). T/NKT, NK-like T cells.

FIG. 3B is a UMAP of cell state abundances (scRNA-seq) versus future irAE status and CD4 TEM cell frequencies (CyTOF). The former was quantified by a two-sided, unpaired Wilcoxon rank-sum test and expressed as −log 10 P values. For associations with no severe irAE, −log 10 P values were multiplied by −1. CD4 T cell states 5 and 3 are indicated together as CD4 T 5+3.

FIG. 3C is a heatmap of DEGs (P_{adj}<0.05) between CD4 T cell states 5 and 3 and other CD4 T cell states. Within each state, the columns represent the mean expression from individual patients converted to z-scores.

FIG. 3D is a set of 2 graphs of (Left) frequencies of candidate activated and resting subsets of CD4 T 5+3 cell states in 13 patients stratified by no severe (n=7) and severe (n=6) irAE status. Activation markers with counts per million (CPM)>0 were considered expressed. Significance was determined by a two-sided, unpaired Wilcoxon rank-sum test, as well as (Right) a receiver operating characteristic curve plot showing the performance of the CD4 T 5+3 subsets (from the left panel) for predicting severe irAE development. NS, not significant.

FIG. 3E is a graph showing pretreatment TCR clonotype diversity within each T cell state, total T cells, CD8 T cells, CD4 T cells, and activated versus resting CD4 T 5+3 cells (defined as in FIG. 3D), grouped by future irAE status. TCR diversity was calculated for all patients with at least 100 TCR clones (n=9). States are ordered by the AUC between TCR diversity and severe irAE status.

FIG. 3F is a color-coded chart of the mean expression of key lineage and activation genes in CD4 T cell states. States within the box are consistent with TEM and TEM-like phenotypes. The box center lines, box bounds, and whiskers indicate the medians, first and third quartiles, and minimum and maximum values, respectively.

FIG. 4A is a graph showing the association between pretreatment peripheral blood leukocyte composition (CIBERSORTx) and severe irAE development in bulk cohort 1 (n=26 patients) and bulk cohort 2 (n=27 patients) (FIG. 1). Significance was determined by two-sided, unpaired Wilcoxon rank-sum test and expressed as −log 10 P values. For associations with no severe irAE, −log 10 P values were multiplied by −1.

FIG. 4B is a graph showing TCR clonotype diversity (Shannon entropy) in both bulk cohorts (n=53 patients), stratified by future irAE status (no severe irAE, n=36; severe irAE, n=17). The box center lines, box bounds, and whiskers denote the medians, first and third quartiles, and minimum and maximum values, respectively. Significance was determined by a two-sided, unpaired Wilcoxon rank-sum test.

FIG. 4C is a graph showing the development of a composite model for the prediction of severe irAEs, integrating activated CD4 TM cell abundance and TCR clonotype diversity from pretreatment peripheral blood transcriptomes, with model scores trained on bulk cohort 1 and shown across both cohorts. The cut-point for high/low scores was optimized using Youden's J statistic on bulk cohort 1.

FIG. 4D is a set of 2 graphs of (Left) a ROC plot showing composite model performance in bulk cohort 2 (held-out validation), whether applied to all patients (both therapies, n=27), combination therapy patients (n=11) or PD-1 monotherapy patients (n=16), as well as (Right) a ROC plot showing composite model performance in bulk cohorts 1 and 2, whether trained on PD-1 patients (n=29) and tested on combination therapy patients (n=24) or vice versa. The AUC is shown for each ROC curve.

FIG. 4E is a graph of composite model scores for all bulk cohort patients (n=53) after model training for severe irAE development with LOOCV (FIG. 13), grouped by the highest irAE grade per patient. The box center lines, box bounds, and whiskers indicate the medians, first and third quartiles, and minimum and maximum values within 1.5× the interquartile range of the box limits, respectively. Statistical significance was determined by a Kruskal-Wallis test.

FIG. 5A is a graph showing pretreatment prediction of time-to-severe irAE onset in patients treated with combination therapy. The cut-point was optimized using composite model scores trained with LOOCV. Only patients from bulk cohorts 1 and 2 who did not experience early progression were analyzed (n=23). Statistical significance was assessed by a two-sided log-rank test.

FIG. 5B is a set of two graphs showing TCR clonal dynamics in relation to severe irAE development in patients treated with combination therapy. Left: Change in TCR clonality from baseline after initiation of combination therapy as measured by 1−Pielou's evenness, with future irAE status indicated by color. Right: Same as the left but showing change in clonality according to future irAE status. Significance was determined by a two-sided, unpaired Wilcoxon rank-sum test.

FIG. 5C is a graph showing enrichment of a CD4 T 5+3 gene signature in CD4 T cells from pretreatment PBMC samples obtained from 3 patients analyzed in FIG. 5B, all of whom developed severe irAEs and showed TCR clonal expansion after ICI initiation (FIG. 15D). The box center lines, box bounds, and whiskers indicate the medians, first and third quartiles and minimum and maximum values, respectively. The points denote cells profiled by scRNA-seq and annotated either by Azimuth (CD4 naive, n=245 cells; CD4 TCM, n=320 cells) or by their clonal persistence from baseline to early on-treatment time points (persistent CD4, n=190 cells). The most persistent CD4 clonotypes in this analysis showed evidence of clonal expansion (FIG. 15F and G). Significance was determined relative to persistent cells by a two-sided, unpaired Wilcoxon rank-sum test. ssGSEA, single-sample GSEA.

FIG. 5D is a graph of the differences in freedom from severe irAE stratified by the degree of TCR clonal expansion after initiating combination therapy, as measured by the change in 1−Pielou's evenness. Patients were grouped into the following tertiles: no clonal expansion (n=5), intermediate (n=5), and high clonal expansion (n=5). Statistical significance was assessed by a two-sided log-rank test.

FIG. 6 is a color-coded chart showing the large-scale assessment of circulating leukocytes in autoimmune diseases. Enrichment of circulating leukocyte levels in two autoimmune disorders relative to healthy controls. Leukocyte composition was determined by CIBERSORTx. Significance was determined by a two-sided, unpaired Wilcoxon rank-sum test and integrative meta z-score. Details of the analytical workflow and underlying datasets are provided in FIG. 16.

FIG. 7A is a UMAP representation of pretreatment peripheral blood leukocytes profiled by droplet-based scRNA-seq (10× Genomics) from 13 patients with metastatic melanoma, colored by major cell lineages, severe irAE status, TCR expression by scV (D) J-seq, and BCR expression by scV (D) J-seq (related to FIG. 3A).

FIG. 7B is a schematic of the unsupervised hierarchical clustering (average linkage) of the mean log 2 transcriptome per CD4 T cell cluster identified from scRNA-seq data.

FIG. 7C is a dot plot showing the average expression of key activation (HLA-DX, MK167) and lineage markers (SELL, CCR7) in CD4 T cell clusters.

FIG. 7D is a graph of the unsupervised hierarchical clustering (average linkage) of the mean log 2 transcriptome per CD4 T cell cluster identified from scRNA-seq data showing all pairwise combinations of scRNA-seq clusters within each of the major cell types analyzed (B cells, CD4 T cells, CD8 T cells, NK cells, monocytes). Across 82 possible pairwise combinations, CD4 T 5+3 achieved the highest Spearman correlation against CD4 TEM levels enumerated by CyTOF and the strongest association with severe irAE development. Cells annotated as ‘T/NKT’ were collapsed into CD8 T cells.

FIG. 7E is a graph of the unsupervised hierarchical clustering (average linkage) of the mean log 2 transcriptome per CD4 T cell cluster identified from scRNA-seq data showing all pairwise combinations ranked by the mean of each feature following unit variance normalization (mean of 0 and standard deviation of 1). In this analysis, the −log 10 P-value for the association with severe irAE (two-sided, unpaired Wilcoxon rank sum test) was normalized to unit variance without considering the direction of the association.

FIG. 8A shows UMAP projections of scRNA-seq data generated in this work, embedded and labeled by Azimuth using a reference PBMC atlas of 162 k cells profiled by scRNA-seq and 228 antibodies.

FIG. 8B is a confusion matrix showing the agreement between phenotypic labels determined by marker genes and unsupervised clustering (rows; related to FIG. 3A and FIG. 7A) versus reference-guided annotation with Azimuth (columns). In total, 85% of single cells assigned to a major lineage group by Azimuth (B cells, CD4 T, CD8 T, NK cells, monocytes) were assigned to the same identity by canonical marker gene assessment. Given the absence of NKT cells in the reference atlas used for Azimuth, the T/NKT cluster defined by unsupervised analysis was relabeled as CD8 T cells.

FIG. 8C is a graph of the same analysis as in FIG. 3B but shown for all 27 phenotypic states identified by Azimuth. Among these states, CD4 TEM was most associated with severe irAE and CyTOF-enumerated CD4 TEM. A population combining CD4 TEM and CD4 Proliferating states was also strongly associated with severe irAE. The latter showed the highest expression of HLA-DX and the lowest expression of SELL (panel d), consistent with an activated CD4 TEM phenotype.

FIG. 8D is a dot plot depicting key activation and lineage markers among CD4 T cell states annotated by Azimuth.

FIG. 8E is a set of violin plots showing protein expression levels imputed by Azimuth using antibody-derived tag (ADT) data, supporting the combination of CD4 TEM and CD4 Proliferating states shown in FIG. 8C and F.

FIG. 8F is a grid showing the performance of top-ranking cell subsets identified by Azimuth and unsupervised clustering for the prediction of severe irAEs. The combined CD4 T 5+3 clusters (FIG. 3B) were more associated with severe irAE and CyTOF than the top-ranking reference-guided population (FIG. 3C). Statistical significance was calculated using a two-sided, unpaired Wilcoxon rank sum test. Data in all panels shown are from the 13 samples profiled by scRNA-seq in FIG. 3.

FIG. 9A is a graph showing the association between severe irAE development and pretreatment levels of T cell states identified by unsupervised clustering (left) and memory-like T cell states identified by Azimuth (right) in 13 PBMC samples profiled by scRNA-seq (FIGS. 1 and 3A). Activated cells were defined as those expressing HLA-DX or MKI67 (CPM>0); resting cells were defined by the absence of HLA-DX and MKI67 expression (CPM=0).

FIG. 9B is a set of graphs showing an analysis of activated, resting, and parental T cell subsets in relation to severe irAE development. Left: Association between severe irAE development and pretreatment levels of memory T cell subsets, total CD4 and CD8 T cells, and total T cells quantified by CyTOF, for all 18 patients analyzed in the single-cell discovery cohort (FIGS. 1 and 2A). Activated phenotypes were defined as CD38+ or HLA-DR+ or Ki67+. Resting phenotypes were defined as CD38−HLA-DR−Ki67−. Right: ROC plot showing the performance of activated and resting CD4 TEM subsets (left panel) for predicting severe irAE development. Cell fractions were assessed relative to total PBMC content. Statistical significance in a, b was determined by a two-sided, unpaired Wilcoxon rank sum test and nominal −log 10 P-values are displayed. −log 10 P-values were further multiplied by −1 for associations with no severe irAE.

FIG. 10A is a schematic showing the key TCR diversity measures and the impact of cell abundance, TCR richness, and distinct clonal repertoires on such measures. Hypothetical CD4 naïve and TEM cell subsets are shown as examples. Triangles depicting differences in magnitude are not drawn to scale.

FIG. 10B is a graph of mean Shannon entropy versus mean clonality (1-Pielou's evenness) for each CD4 T cell state identified by unsupervised clustering of scRNA-seq data. CD4 T 5+3 (FIG. 3B and C), a TEM state enriched for activated cells, shows elevated clonality relative to other CD4 states, as expected for this phenotype, while also showing higher diversity (Shannon entropy), indicating elevated richness.

FIG. 10C is a schematic showing the distribution of EM-like CD4 T cell states (from FIG. 3F) with available scTCR clonotype data.

FIG. 10D is a set of graphs showing the association between severe irAE development and TCR diversity (Shannon entropy) in pseudo-bulk T cells from pretreatment blood, shown for all T cell states identified by scRNA-seq (left) and after the removal of the EM-like states indicated in FIG. 10C (no severe irAE, n=5 patients; severe irAE, n=4 patients). Bounds of the box and whiskers indicate medians, 1st and 3rd quartiles, and minimum and maximum values, respectively.

FIG. 10E is a graph showing the same association as in FIG. 10D but shown for EM-like states alone. Bounds of the box and whiskers indicate medians, 1st and 3rd quartiles, and minimum and maximum values, respectively.

FIG. 10F is a graph showing the area under the curve (AUC) for the association between pretreatment peripheral TCR diversity (Shannon entropy) and severe irAE development, shown for all combinations of the constituent cell states in e, including the combined CD4 T 5+3 cluster after restricting to activated cells (CPM>0 for HLA-DX or MKI67). Of note, no other combination of activated EM-like states achieved an AUC >0.85 in this analysis.

FIG. 10G is a graph showing BCR clonotype diversity (Shannon entropy), shown for each B cell state identified by unsupervised clustering (FIG. 3A). In FIG. 10B and D-F, only patients with at least 100 TCR clones were analyzed (n=9). The same patients were analyzed in FIG. 10G for consistency. Bounds of the box and whiskers indicate medians, 1st and 3rd quartiles, and minimum and maximum values, respectively.

FIG. 11A is a graph showing the expression of developmentally-regulated marker genes in major CD4 T cell subsets from the LM22 signature matrix (MAS5 normalized), showing that the LM22 reference signature for activated CD4 memory T cells has a TEM profile.

FIG. 11B is a graph showing CIBERSORTx versus mass cytometry for the enumeration of activated CD4 memory T cells in the pretreatment peripheral blood of 17 metastatic melanoma patients. A linear regression line with 95% confidence band is shown. Concordance and significance were determined by Pearson r and a two-sided t-test, respectively. While activated CD4 memory T cells quantitated by CyTOF were defined by CD38 expression in this plot, other activated CD4 TEM subsets were also significantly correlated with CIBERSORTx (FIG. 11C).

FIG. 11C is a cross-correlation plot of lymphocyte subset frequencies determined by CyTOF and CIBERSORTx. Act., Activated.

FIG. 11D is a cross-correlation plot showing the correlation between activated CD4 memory T cell levels inferred by CIBERSORTx and 14 memory T cell states profiled by CyTOF, including CD38+ activated subsets manually gated within each population, in PBMCs from 17 metastatic melanoma patients.

FIG. 11E is a scatter plot depicting the global correlation of lymphocyte subsets enumerated by CIBERSORTx and flow cytometry in peripheral blood samples from five healthy subjects profiled by bulk RNA-seq. A linear regression line with 95% confidence band is shown. Concordance and significance were determined by Pearson r and a two-sided t-test, respectively. As monocytes were variably underestimated by cytometry compared to complete blood counts, all results in b-e are expressed as a function of total lymphocytes.

FIG. 11F is a graph showing the distribution of activated CD4 memory T cell levels quantitated by CyTOF (CD38+, HLA-DR+ or Ki67+CD4 TEM cells, n=28 patients), scRNA-seq (HLA-DX+ or MKI67+ cells within CD4 T clusters 5 and 3, n=13 patients), and CIBERSORTx (n=60 patients) across all irAE-evaluable samples profiled by each modality in this work. Box center lines, bounds of the box, and whiskers indicate medians, 1st and 3rd quartiles, and minimum and maximum values, respectively. Statistical significance was determined by a Kruskal-Wallis test. n.s., not significant (P>0.05).

FIG. 12A is a graph showing an association between baseline bulk TCR diversity and the highest irAE grade observed for each patient in bulk cohorts 1 and 2, shown for Shannon entropy and stratified by therapy type. Patients treated with combination therapy are stratified by future irAE status: no severe irAE (n=10) versus severe irAE (n=14 patients) (left) and irAE grade (right): 0/1 (n=3), 2 (n=7), 3 (n=12), and 4 (n=2). Two-group comparisons were assessed by a two-sided, unpaired Wilcoxon rank sum test. n.s., not significant (P>0.05). Linear regression was applied to evaluate the median value of each measure grouped by irAE grade (insets). The significance of linear concordance was determined by a two-sided t-test. Grades 0 and 1 reflect no toxicity and asymptomatic toxicity, respectively, and were combined. The box center lines, bounds of the box, and whiskers denote medians, 1st and 3rd quartiles, and minimum and maximum values within 1.5×IQR (interquartile range) of the box limits, respectively.

FIG. 12B is a graph showing the association between baseline bulk TCR diversity and the highest irAE grade observed for each patient in bulk cohorts 1 and 2, shown for the Gini-Simpson index and stratified by therapy type. Patients treated with combination therapy are stratified by future irAE status: no severe irAE (n=10) versus severe irAE (n=14 patients) (left) and irAE grade (right): 0/1 (n=3), 2 (n=7), 3 (n=12), and 4 (n=2). Two-group comparisons were assessed by a two-sided, unpaired Wilcoxon rank sum test. n.s., not significant (P>0.05). Linear regression was applied to evaluate the median value of each measure grouped by irAE grade (insets). The significance of linear concordance was determined by a two-sided t-test. Grades 0 and 1 reflect no toxicity and asymptomatic toxicity, respectively, and were combined. The box center lines, bounds of the box, and whiskers denote medians, 1st and 3rd quartiles, and minimum and maximum values within 1.5×IQR (interquartile range) of the box limits, respectively.

FIG. 12C is a graph showing the association between baseline bulk TCR diversity and the highest irAE grade observed for each patient in bulk cohorts 1 and 2, shown for Shannon entropy and stratified by therapy type. Patients treated with PD1 monotherapy are stratified by future irAE status: no severe irAE (n=26) versus severe irAE (n=3 patients) (left) and irAE grade (right): 0/1 (n=19), 2 (n=7), 3 (n=2), and 4 (n=1). Two-group comparisons were assessed by a two-sided, unpaired Wilcoxon rank sum test. n.s., not significant (P>0.05). Linear regression was applied to evaluate the median value of each measure grouped by irAE grade (insets). The significance of linear concordance was determined by a two-sided t-test. Grades 0 and 1 reflect no toxicity and asymptomatic toxicity, respectively, and were combined. The box center lines, bounds of the box, and whiskers denote medians, 1st and 3rd quartiles, and minimum and maximum values within 1.5×IQR (interquartile range) of the box limits, respectively.

FIG. 12D is a graph showing the association between baseline bulk TCR diversity and the highest irAE grade observed for each patient in bulk cohorts 1 and 2, shown for the Gini-Simpson index and stratified by therapy type. Two-group comparisons were assessed by a two-sided, unpaired Wilcoxon rank sum test. n.s., not significant (P>0.05). Linear regression was applied to evaluate the median value of each measure grouped by irAE grade (insets). The significance of linear concordance was determined by a two-sided t-test. Grades 0 and 1 reflect no toxicity and asymptomatic toxicity, respectively, and were combined. The box center lines, bounds of the box, and whiskers denote medians, 1st and 3rd quartiles, and minimum and maximum values within 1.5×IQR (interquartile range) of the box limits, respectively.

FIG. 13A is a graph similar to that seen in FIG. 4D, but applied to both bulk cohorts (n=53 patients) using leave-one-out cross-validation (LOOCV).

FIG. 13B is a graph similar to that seen in FIG. 4C, but shown for model scores determined by LOOCV.

FIG. 13C is a plot showing the performance of the composite model versus other candidate pretreatment factors for predicting severe irAE development. The composite model was trained in bulk cohort 1 (BC1) and validated in bulk cohort 2 (BC2) or vice versa, as indicated.

FIG. 13D is a graph showing the performance of the composite model trained on bulk cohort 1 for predicting severe irAEs in different patient subgroups from bulk cohort 2. DCB, durable clinical benefit; NDB, no durable clinical benefit; GI, gastrointestinal.

FIG. 13E is a graph showing composite model scores determined by LOOCV for all bulk cohort patients treated with combination therapy (n=24), stratified by future irAE grade: 0/1 (n=3), 2 (n=7), 3 (n=12), and 4 (n=2). Center lines, bounds of the box, and whiskers indicate medians, 1st and 3rd quartiles, and minimum and maximum values within 1.5×IQR (interquartile range) of the box limits, respectively. Statistical significance was determined by a Kruskal-Wallis test.

FIG. 13F is a graph showing model performance for predicting grade 2+, 3+, or 4 irAE development in combination therapy patients using the scores in FIG. 13E.

FIG. 13G is a graph showing composite model scores determined by LOOCV in both bulk cohorts (n=53 patients) versus the number of symptomatic irAEs (grade 2+) per patient. Center lines, bounds of the box, and whiskers indicate medians, 1st and 3rd quartiles, and minimum and maximum values within 1.5×IQR (interquartile range) of the box limits, respectively. Statistical significance was determined by a Kruskal-Wallis test.

FIG. 13H is a graph showing composite model scores determined by LOOCV in both bulk cohorts (n=53 patients) versus the number of organ system toxicities per patient. Center lines, bounds of the box, and whiskers indicate medians, 1st and 3rd quartiles, and minimum and maximum values within 1.5×IQR (interquartile range) of the box limits, respectively. Statistical significance was determined by a Kruskal-Wallis test.

FIG. 13I is a plot showing the distribution of irAEs across patients and organ systems. Patients from bulk cohorts 1 and 2 are organized by decreasing composite model scores determined via LOOCV. The line distinguishing high/low scores was optimized using LOOCV.

FIG. 13J is a graph showing the fraction of patients in both bulk cohorts that developed irAEs in at least 2 organ systems versus those that did not, stratified by the threshold in FIG. 13I. Significance was determined by a two-sided Fisher's exact test.

FIG. 14 is a set of graphs showing composite model performance for predicting time to severe irAE in validation bulk cohort 2.

FIG. 14A is a graph showing a-c, Kaplan-Meier analysis for freedom from severe irAE in bulk cohort 2 for patients treated with combination or PD1 immune checkpoint blockade (a), combination therapy (b), or PD1 monotherapy (c), stratified by the composite model score. Statistical significance was calculated by a two-sided log-rank test. In all panels, training was performed in bulk cohort 1, and the cut-point predicting severe irAE was optimized for bulk cohort 1 using Youden's J statistic. Notably, the analyses in a-c were landmarked between treatment initiation and three months following treatment initiation, with all severe irAEs occurring within this period. The Kaplan-Meier plots are shown out to four months given the extended follow-up of patients that did not develop any severe irAE.

FIG. 14A is a graph showing Kaplan-Meier analysis for freedom from severe irAE in bulk cohort 2 for patients treated with combination or PD1 immune checkpoint blockade, stratified by the composite model score. Statistical significance was calculated by a two-sided log-rank test. In all panels, training was performed in bulk cohort 1, and the cut-point predicting severe irAE was optimized for bulk cohort 1 using Youden's J statistic. Notably, the analyses were landmarked between treatment initiation and three months following treatment initiation, with all severe irAEs occurring within this period. The Kaplan-Meier plots are shown out to four months given the extended follow-up of patients that did not develop any severe irAE.

FIG. 14B is a graph showing Kaplan-Meier analysis for freedom from severe irAE in bulk cohort 2 for patients treated with combination therapy, stratified by the composite model score. Statistical significance was calculated by a two-sided log-rank test. In all panels, training was performed in bulk cohort 1, and the cut-point predicting severe irAE was optimized for bulk cohort 1 using Youden's J statistic. Notably, the analyses in FIG. 14A-C were landmarked between treatment initiation and three months following treatment initiation, with all severe irAEs occurring within this period. The Kaplan-Meier plots are shown out to four months given the extended follow-up of patients that did not develop any severe irAE.

FIG. 14C is a graph showing Kaplan-Meier analysis for freedom from severe irAE in bulk cohort 2 for patients treated with PD1 monotherapy, stratified by the composite model score. Statistical significance was calculated by a two-sided log-rank test. In all panels, training was performed in bulk cohort 1, and the cut-point predicting severe irAE was optimized for bulk cohort 1 using Youden's J statistic. Notably, the analyses in a-c were landmarked between treatment initiation and three months following treatment initiation, with all severe irAEs occurring within this period. The Kaplan-Meier plots are shown out to four months given the extended follow-up of patients that did not develop any severe irAE.

FIG. 15A is a graph showing evenness (Pielou's index) of TCR repertoires assembled by MiXCR (bulk RNA-seq) and immunoSEQ® (genomic DNA) from paired pretreatment PBMC samples (n=15 combination therapy patients). Concordance and significance were determined by Spearman p and a two-sided t-test, respectively.

FIG. 15B is a graph similar to that in FIG. 5B but showing clonality for each pre- and on-treatment PBMC sample. Statistical significance was determined by a two-sided, paired Wilcoxon rank sum test. ns, not significant (P>0.05).

FIG. 15C is a graph showing the fraction of pretreatment peripheral blood TCR clonotypes detected on-treatment in 15 combination therapy patients, stratified by no severe (n=6) and severe (n=9) irAE status. Clonotypes with matching productive CDR3 β-chain nucleotide sequences were considered identical. Center lines, bounds of the box, and whiskers indicate medians, 1st and 3rd quartiles, and minimum and maximum values, respectively. Significance was determined by a two-sided, unpaired Wilcoxon rank sum test.

FIG. 15D is a schematic showing persistent T cell clones identified by immunoSEQ® were cross-referenced with scTCR-seq and scRNA-seq data of pretreatment PBMCs from the same three patients (YUALOE, YUNANCY, YUHONEY), all of whom received combination therapy and developed severe ICI-induced toxicity.

FIG. 15E is a dot plot showing log 2 expression of key lineage and activation markers across major T cell states annotated by Azimuth along with persistent clones classified into CD4 and CD8 T cells.

FIG. 15F is a graph showing an aggregate change from baseline in the productive frequencies of persistent clonotypes, stratified by lineage (n=2 cell types) and patient (n=3). The sum of the difference in productive frequencies (on-treatment %-pretreatment %) was calculated from immunoSEQ® data. Bars denote mean+/−SD.

FIG. 15G is a set of graphs showing peripheral blood TCR-β profiling with immunoSEQ®. Top: Change in bulk TCR clonality from baseline (FIG. 5b). Bottom: Same as FIG. 15F but showing the underlying clonotypes, where circle size is proportional to pretreatment clone frequency (immunoSEQ®).

FIG. 15H is a graph similar to that seen in FIG. 5D but restricted to blood draws taken cycle 1 day 1 of combination therapy and <1 month later (n=7 patients).

FIG. 16 is a schema of a large-scale assessment of peripheral blood leukocytes in autoimmune disorders versus healthy controls. Schema describing the workflow and statistical meta-analysis for evaluating the enrichment of individual circulating leukocyte subsets in autoimmune disorders relative to healthy controls (FIG. 6). In brief, CIBERSORTx was applied to enumerate 15 leukocyte subsets in bulk RNA-seq or microarray profiles of peripheral blood samples from patients with either systemic lupus erythematosus57-59 (SLE; n=239) or inflammatory bowel disease (IBD; n=348) compared to healthy controls. For each dataset and cell subset, a two-sided, unpaired Wilcoxon rank sum test was applied to assess the difference in relative abundance between healthy and disease phenotypes. Results were subsequently combined across studies by meta-z statistics (Meth

FIG. 17A is a schematic showing a-e, Gating hierarchies and staining results for CD4 T cell subsets and NKT cells profiled by CyTOF from pretreatment PBMCs. All CD4 T cell subsets except T regulatory cells (Tregs) were gated analogously for CD8 T cells. TCM, central memory T cell; TEM, effector memory T cell; EMRA, CD45RA+ terminally differentiated effector memory T cell.

FIG. 17B is a schematic showing gating hierarchies and staining results for activated vs. resting CD4 TEM cells profiled by CyTOF from pretreatment PBMCs.

FIG. 17C is a schematic showing gating hierarchies and staining results for monocyte subsets profiled by CyTOF from pretreatment PBMCs.

FIG. 17D is a schematic showing gating hierarchies and staining results for B cell subsets profiled by CyTOF from pretreatment PBMCs.

FIG. 17E is a schematic showing gating hierarchies and staining results for NK cell subsets profiled by CyTOF from pretreatment PBMCs.

FIG. 18 is a set of graphs showing a comparison of automated and manual cell state quantitation from CyTOF data.

FIG. 18A is a scatter plot showing the concordance in frequencies between automated gating (Astrolabe) and manual gating for the indicated peripheral blood cell types. Concordance was assessed by Pearson correlation and linear regression (95% confidence band is shown). A two-sided t-test was used to assess statistical significance. Data are from patients analyzed by CyTOF in FIG. 1 (n=18).

FIG. 18B is a scatter plot similar to that seen in FIG. 18A but for CD4 TEM cells. A representative gating scheme for CD4 TEM is provided in FIG. 7A. Concordance was assessed by Pearson correlation and linear regression (95% confidence band is shown). A two-sided t-test was used to assess statistical significance. Data in are from patients analyzed by CyTOF in FIG. 1 (n=18).

FIG. 18C is a graph showing the association of pretreatment CD4 TEM abundance with severe irAE development when expressed as a fraction of total PBMCs, total T cells, or CD4 T cells. Box center lines, bounds of the box, and whiskers denote medians, 1st and 3rd quartiles, and minimum and maximum values, respectively. Data in are from patients analyzed by CyTOF in FIG. 1 (n=18).

FIG. 19 is a schematic representing the methods described in the current disclosure.

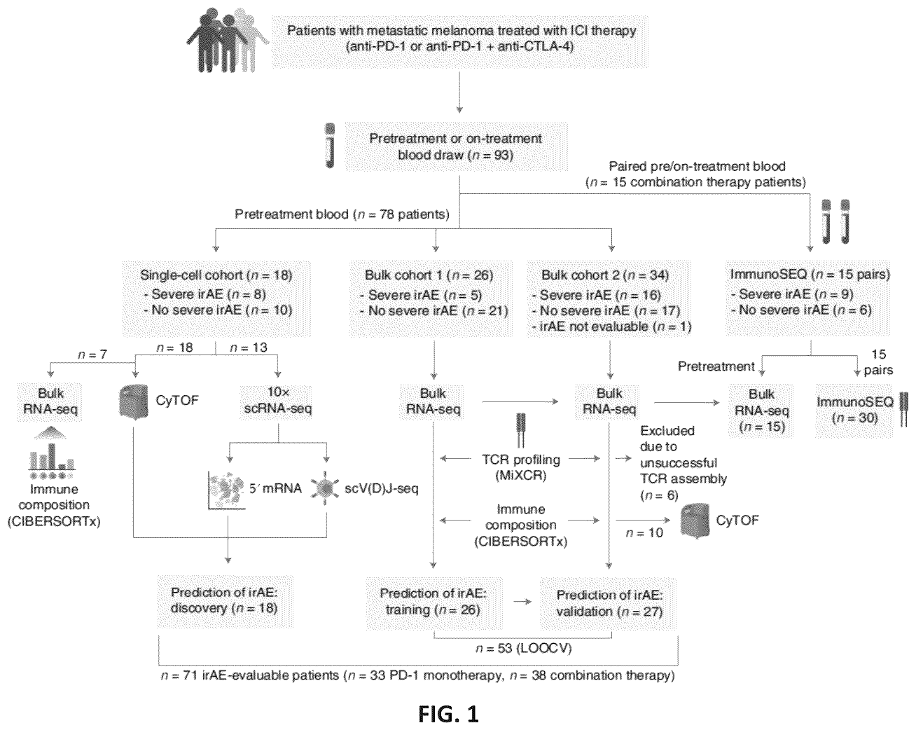

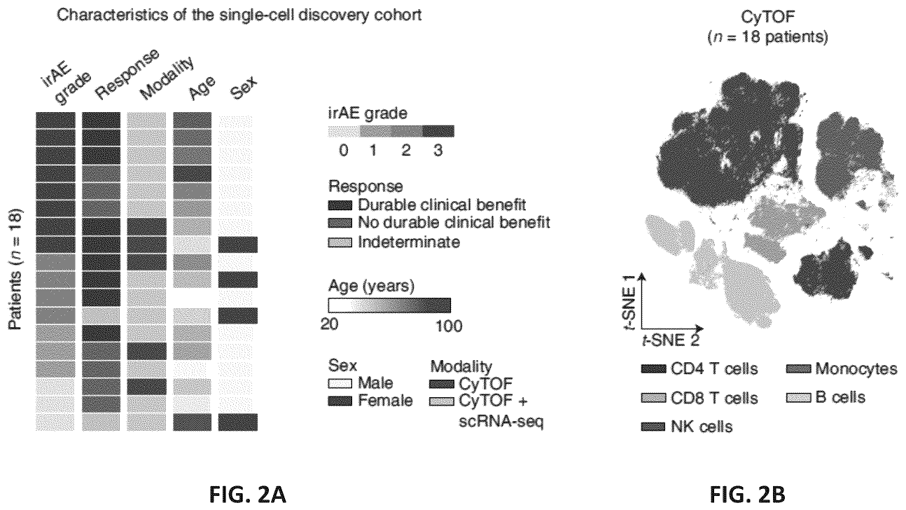

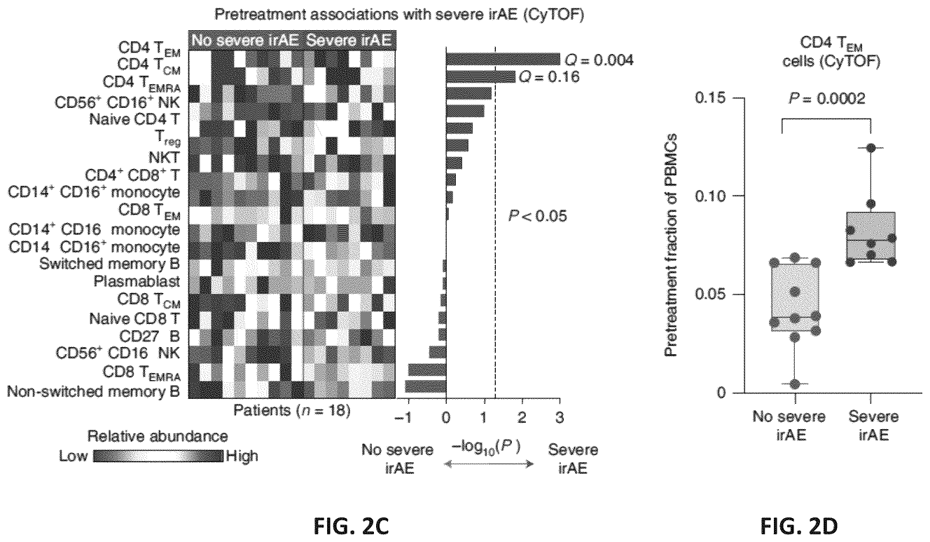

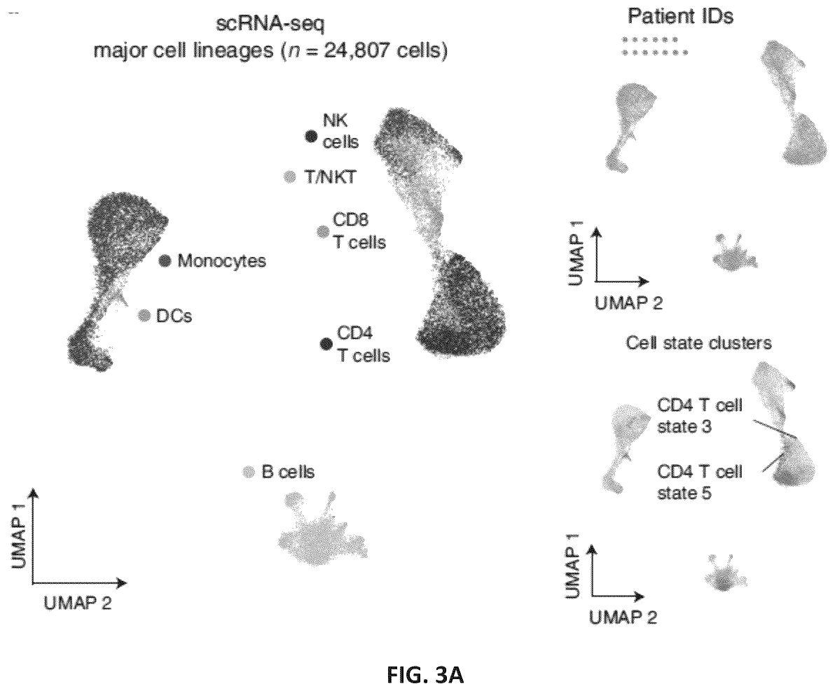

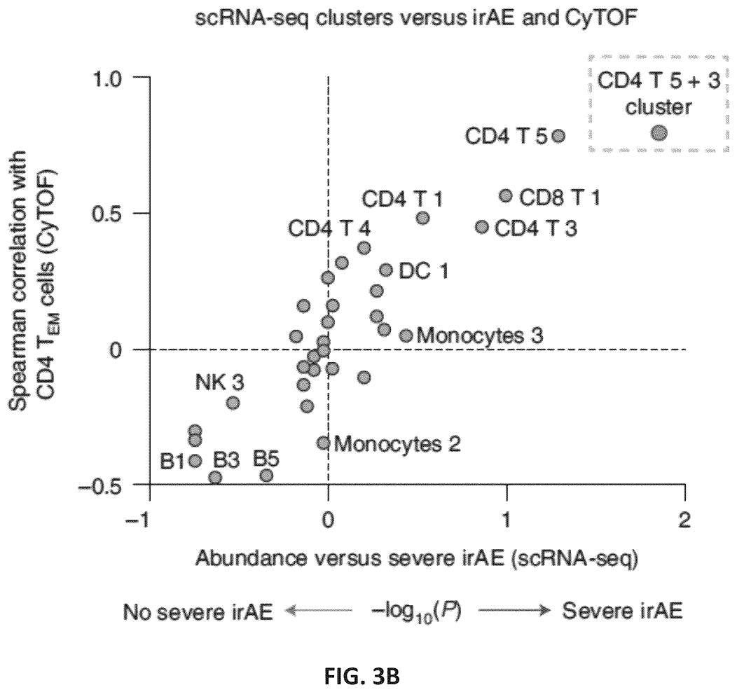

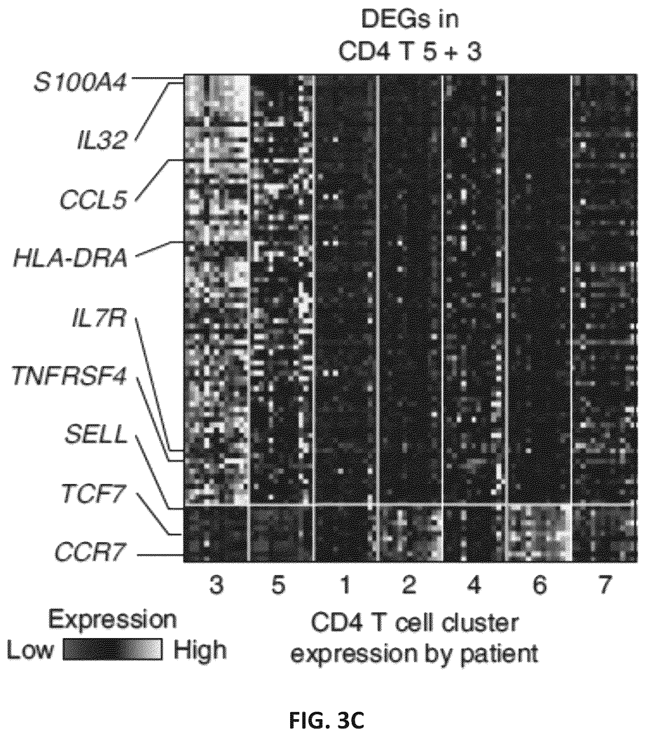

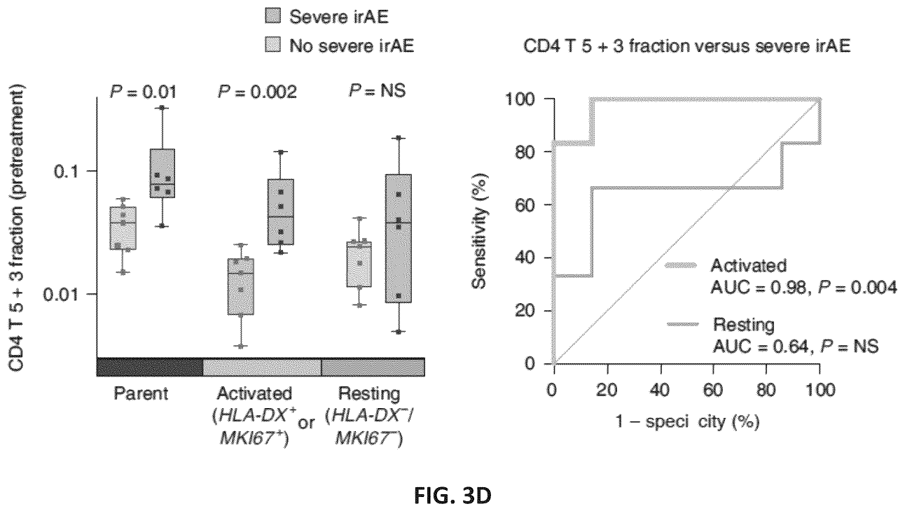

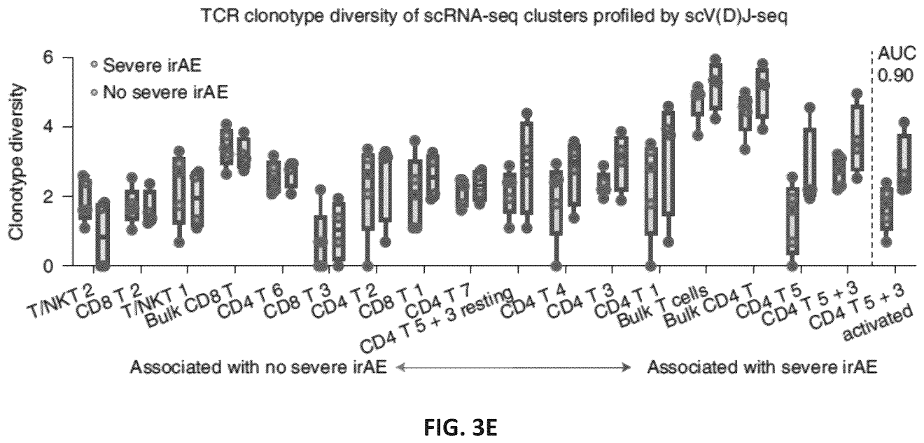

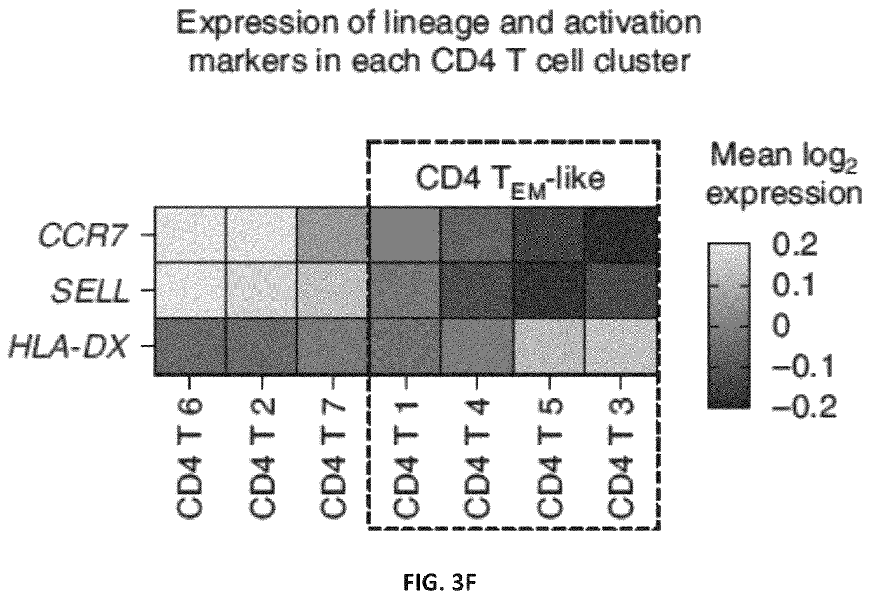

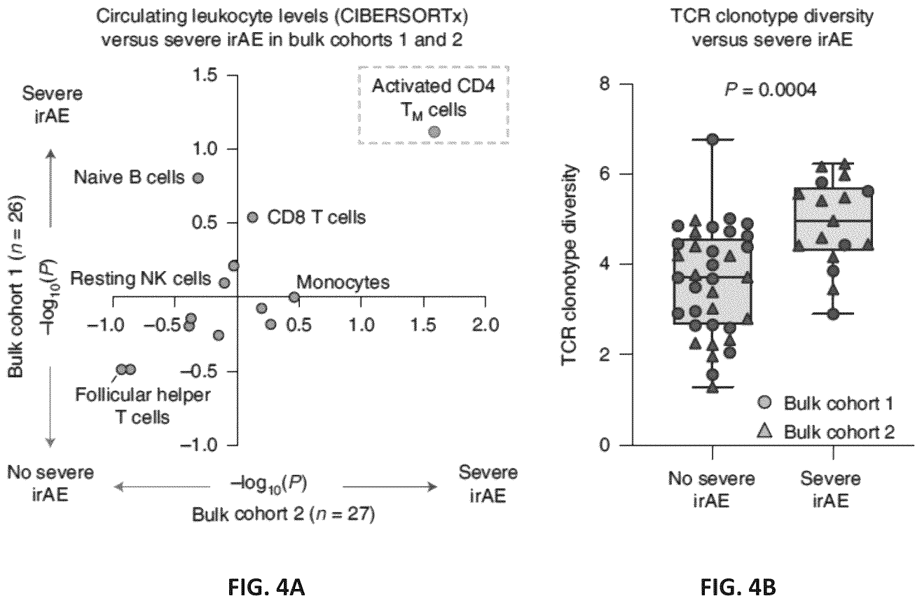

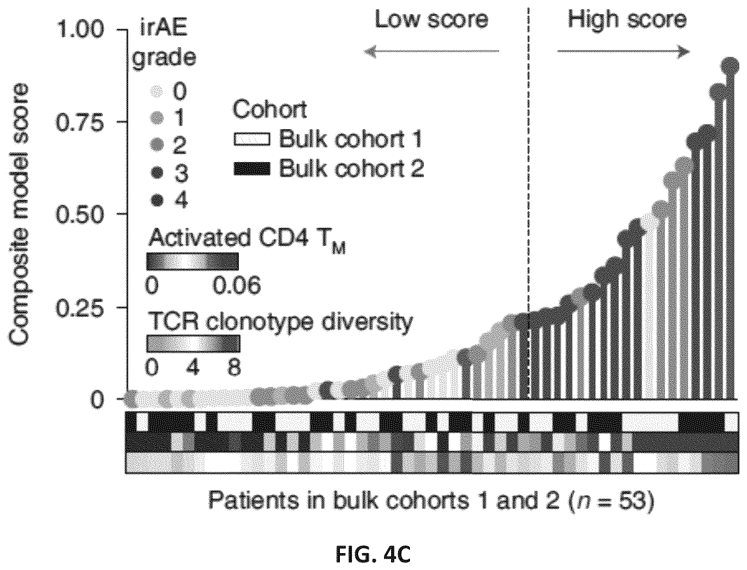

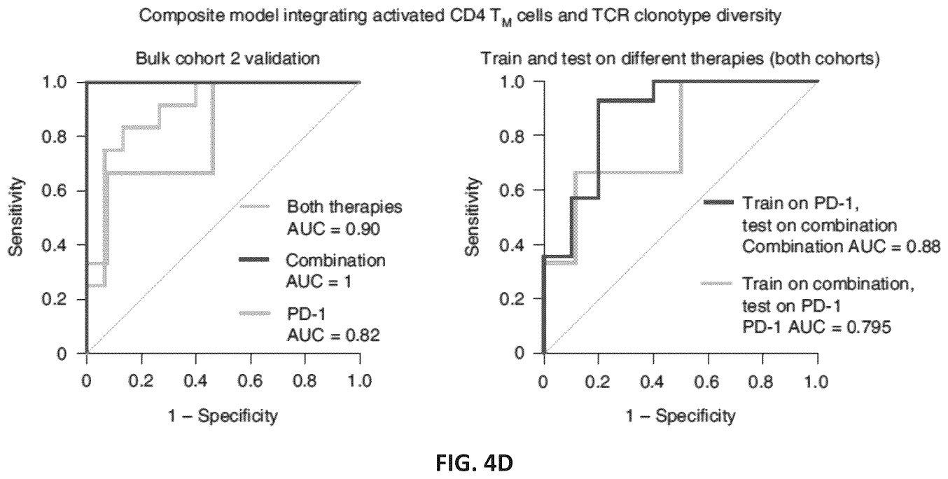

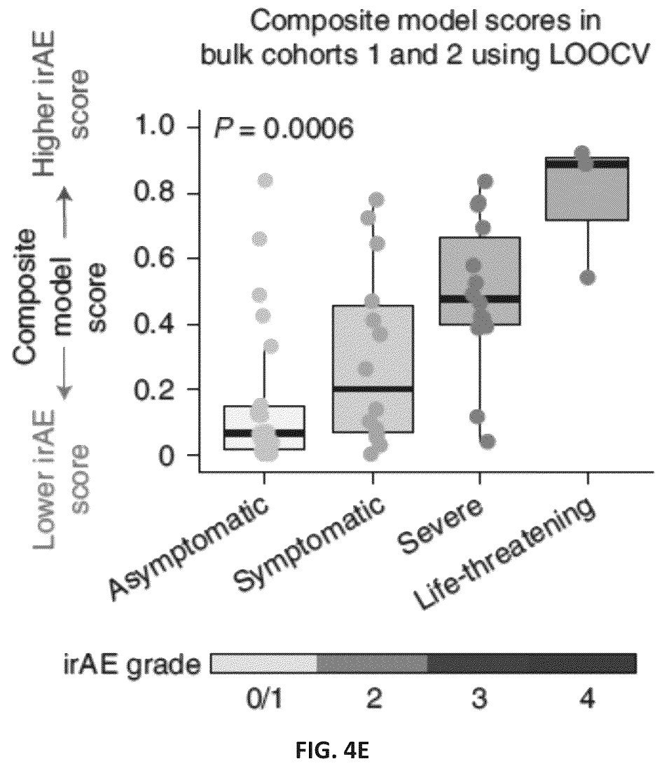

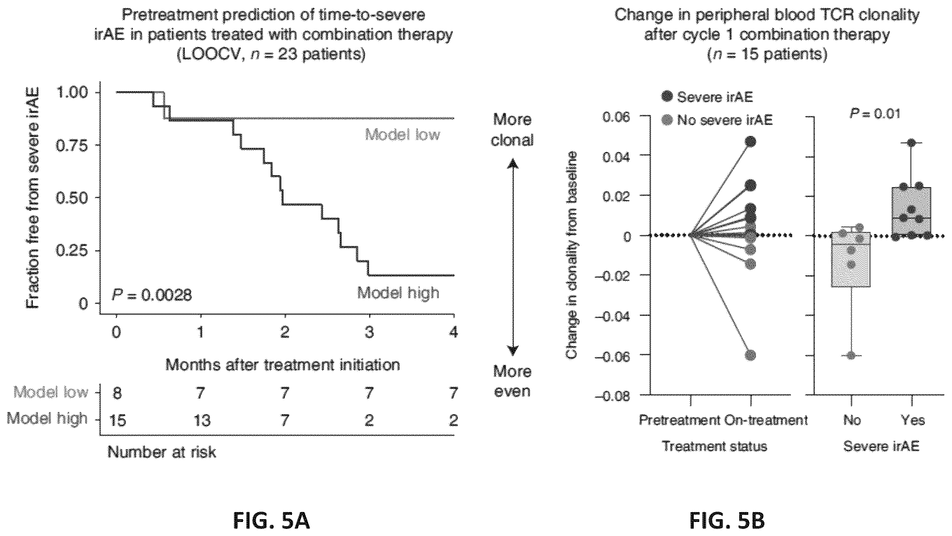

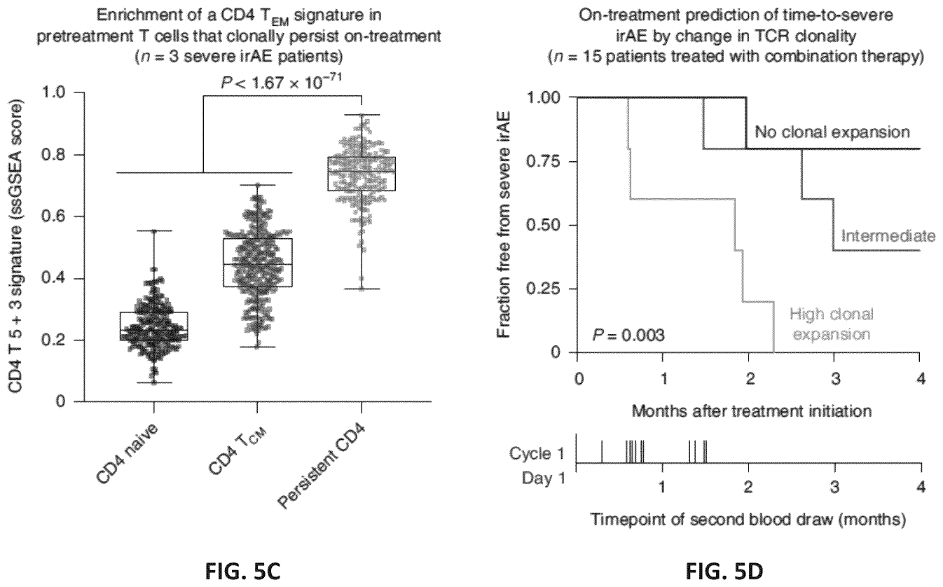

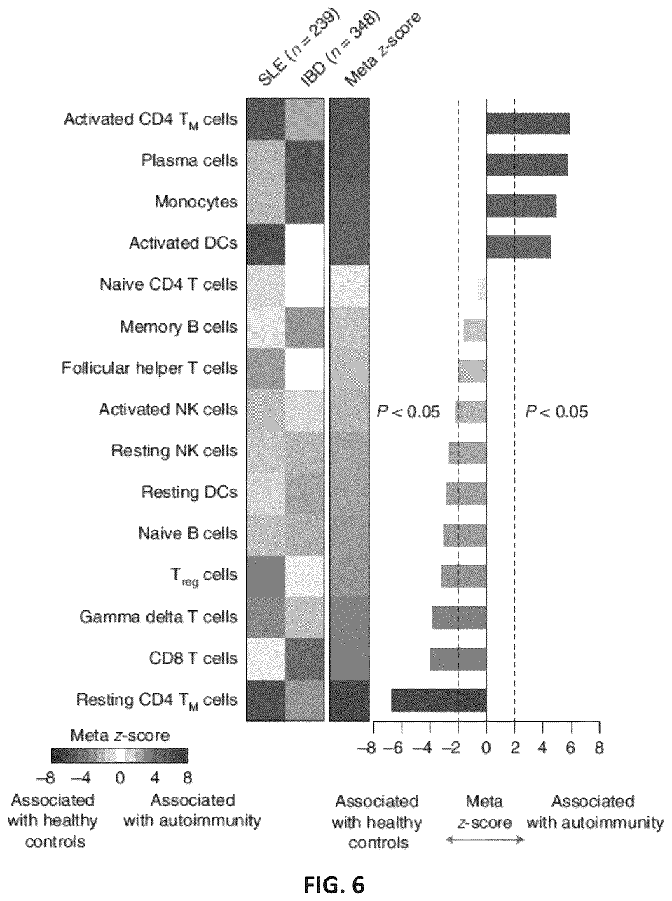

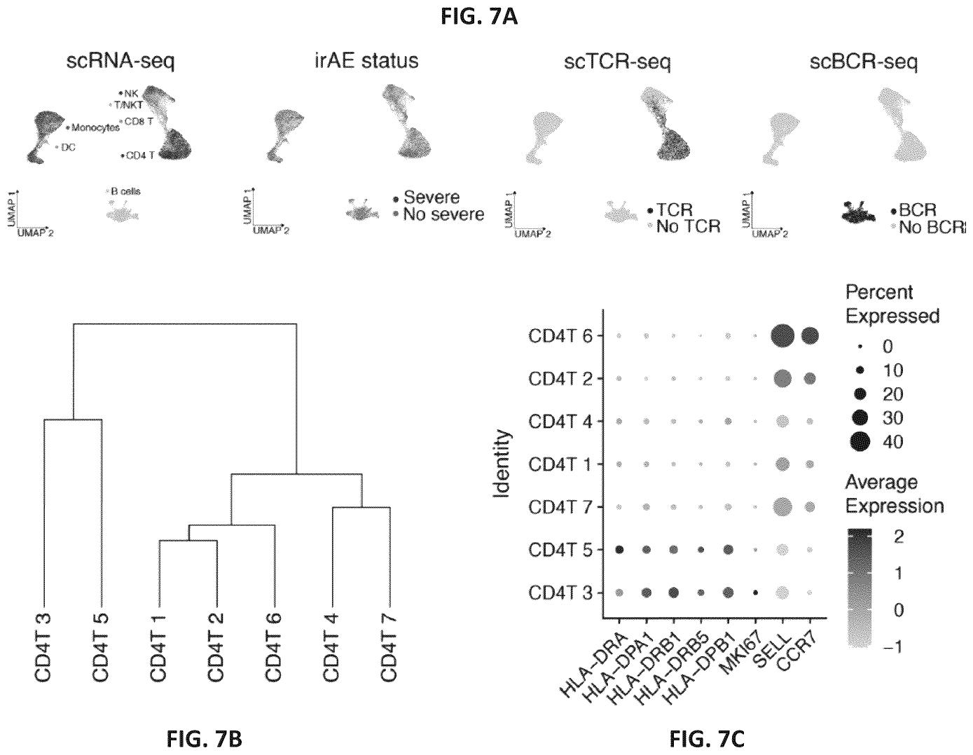

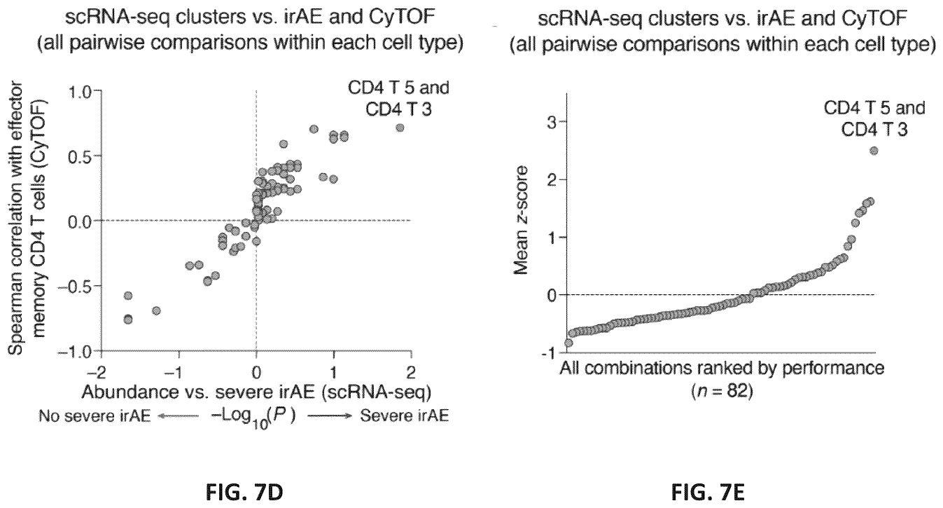

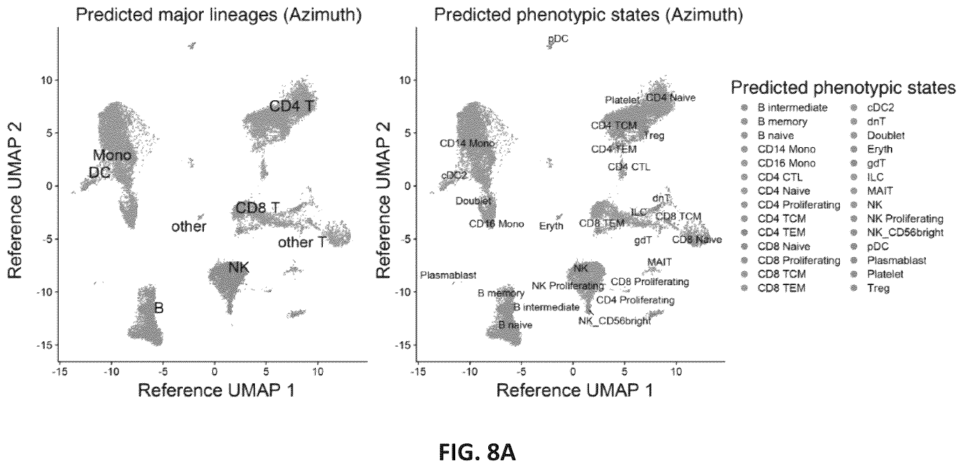

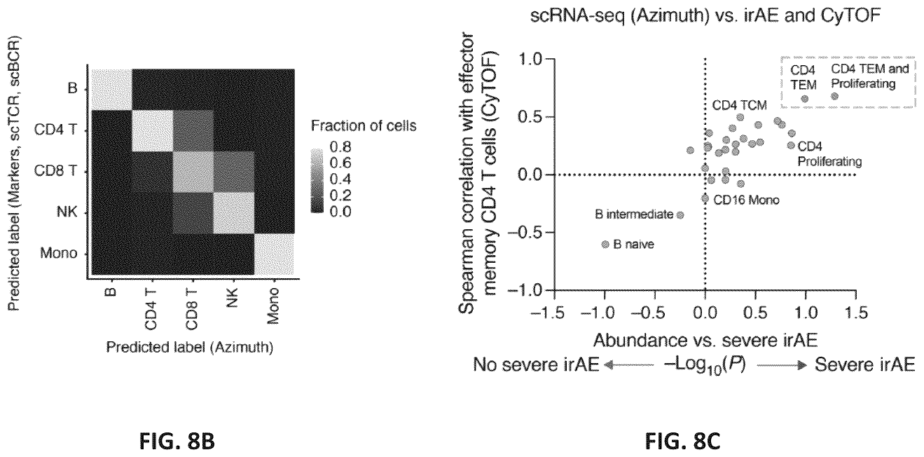

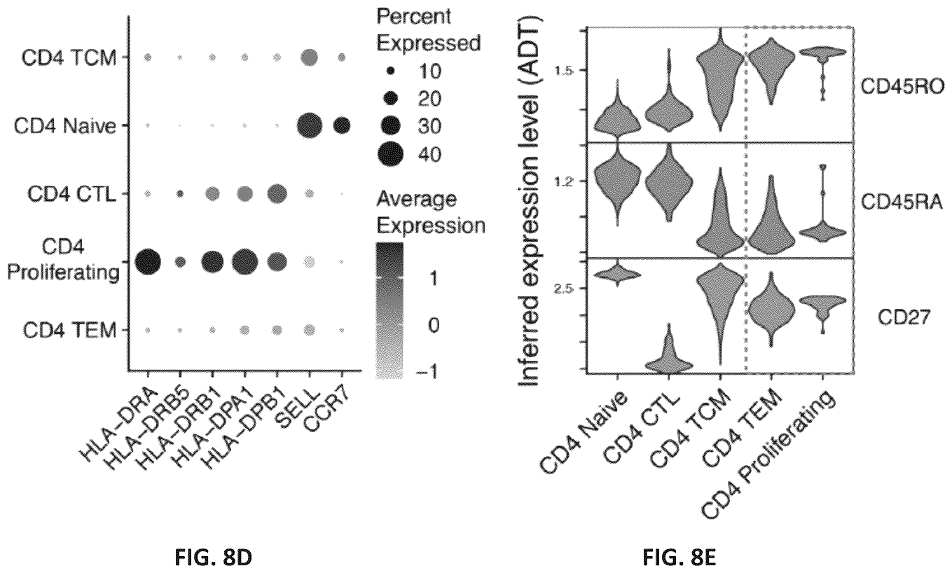

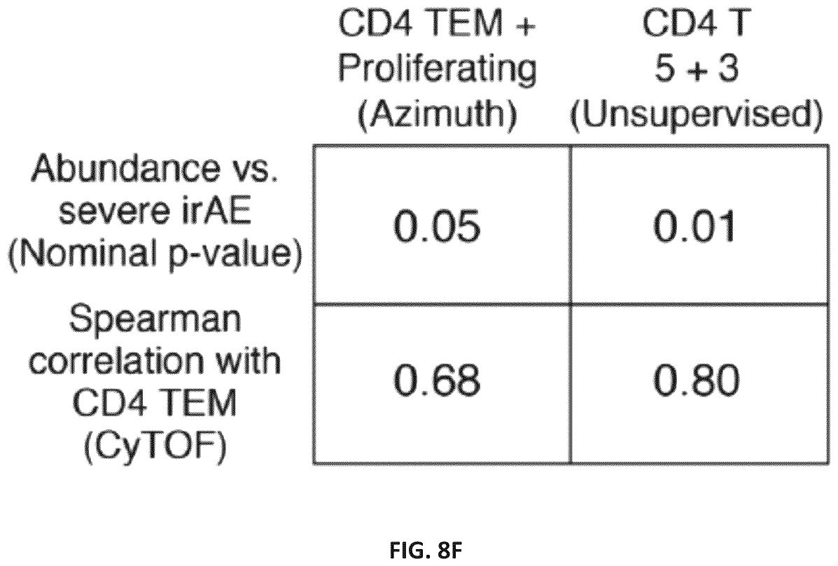

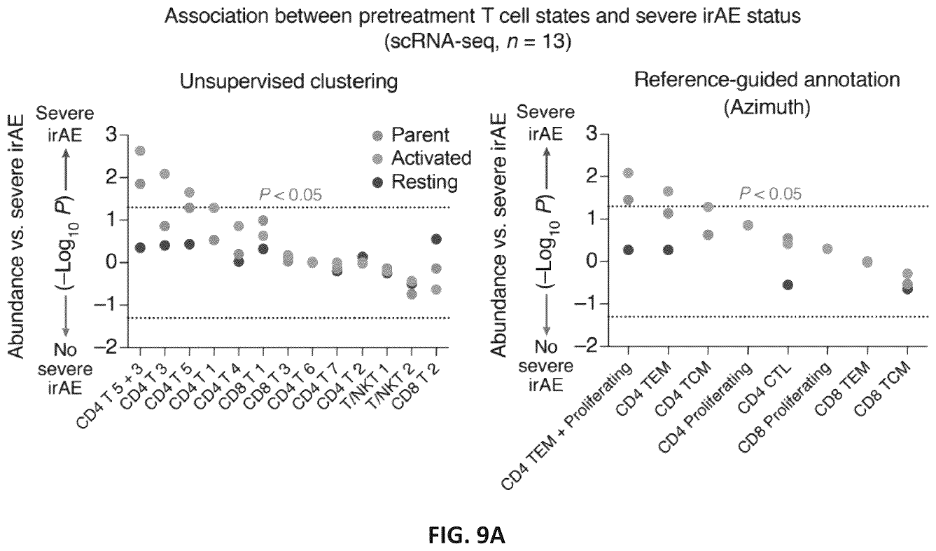

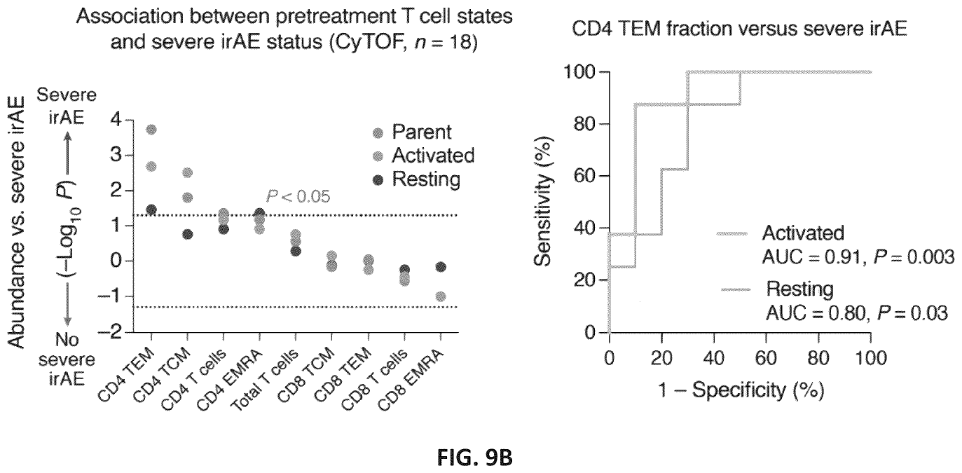

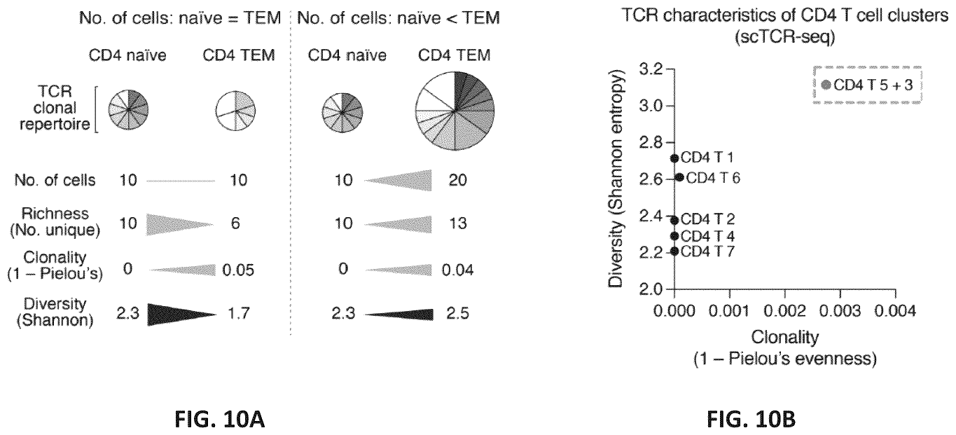

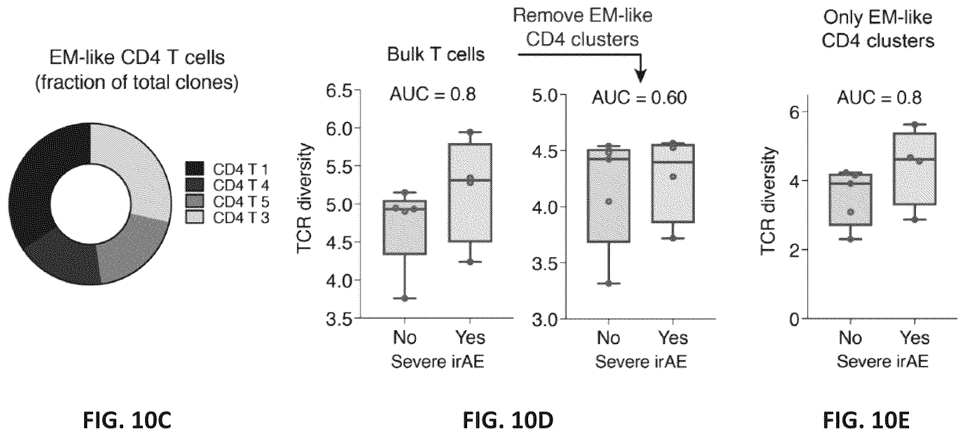

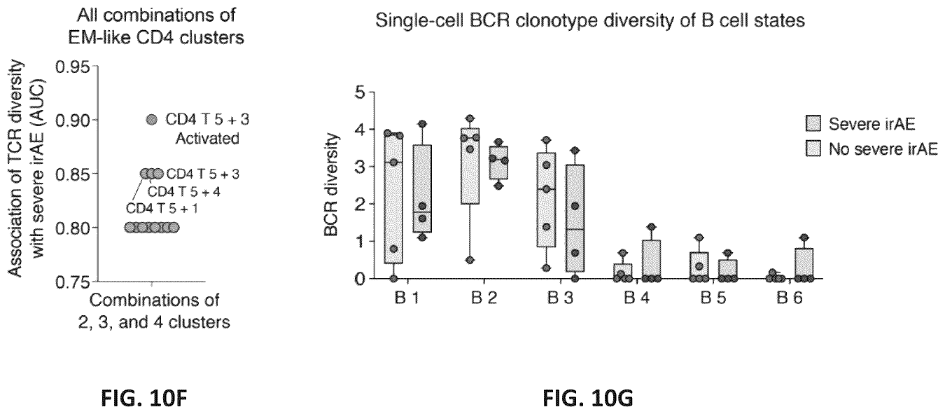

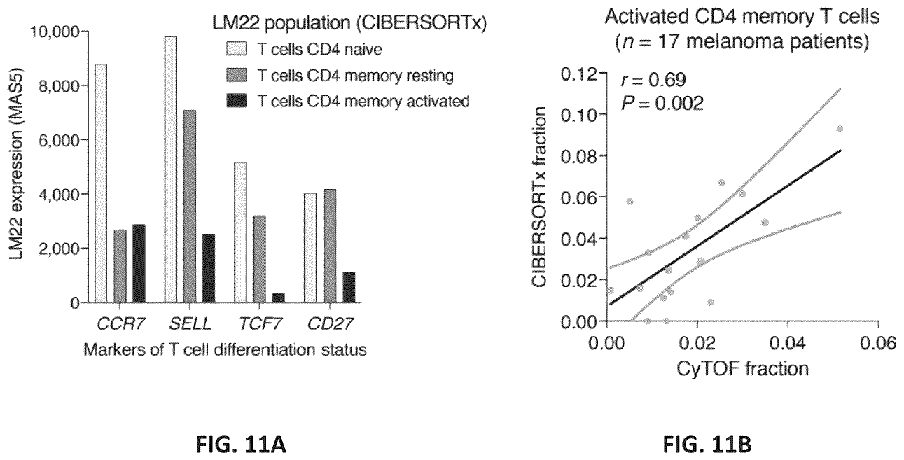

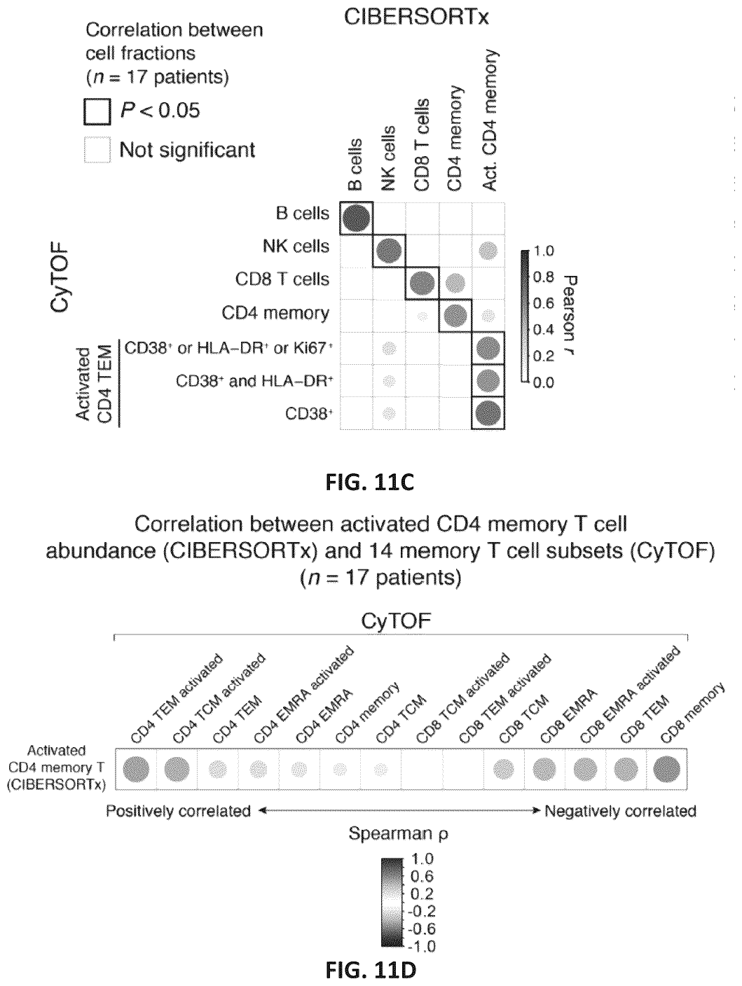

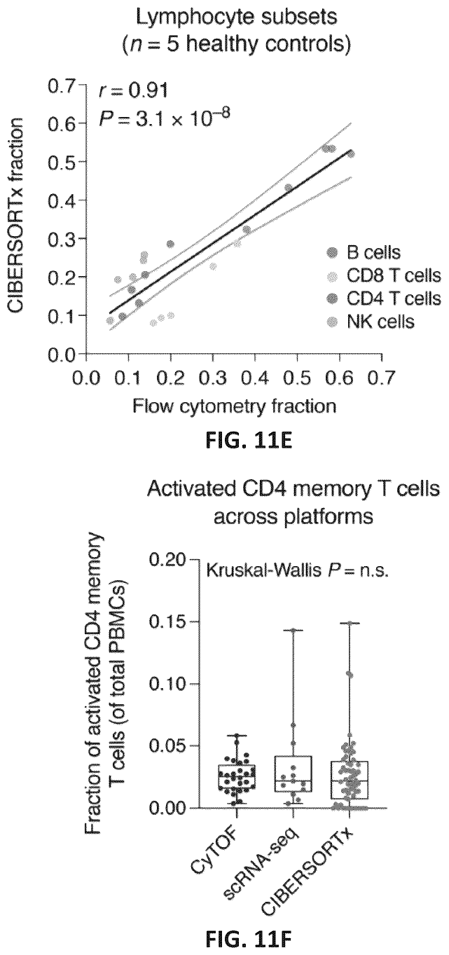

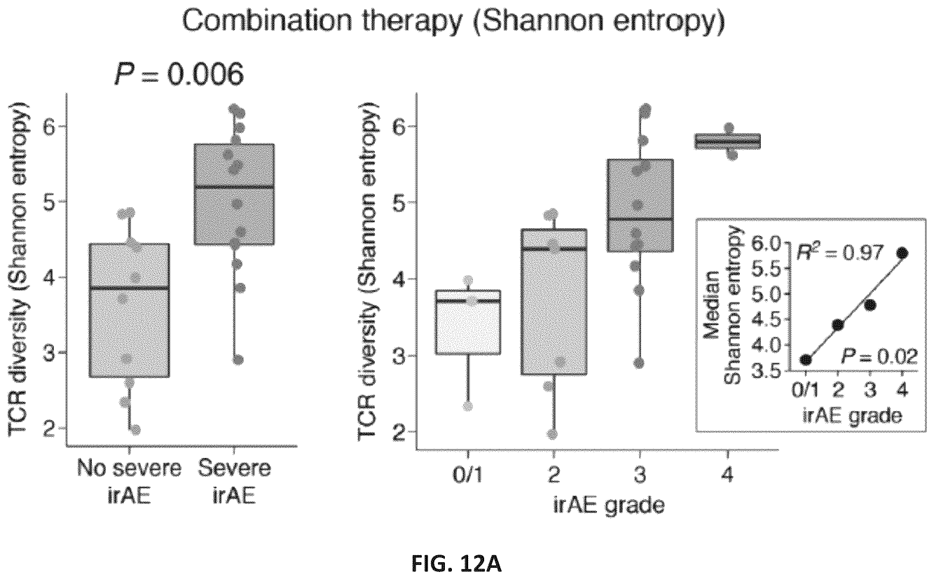

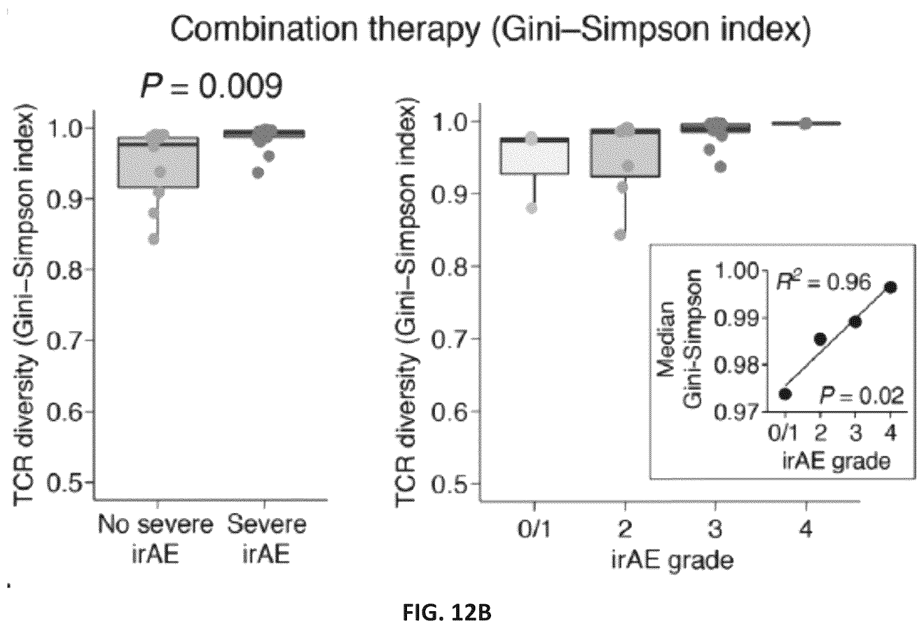

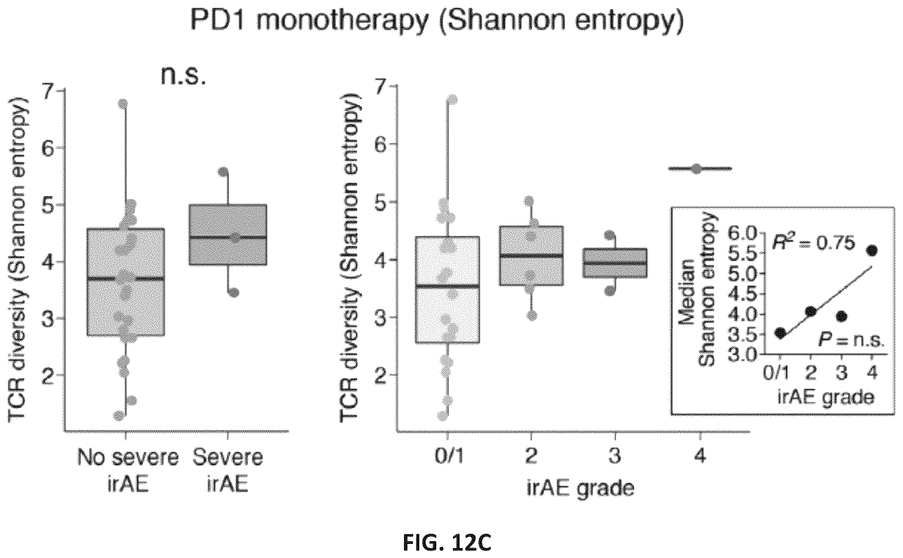

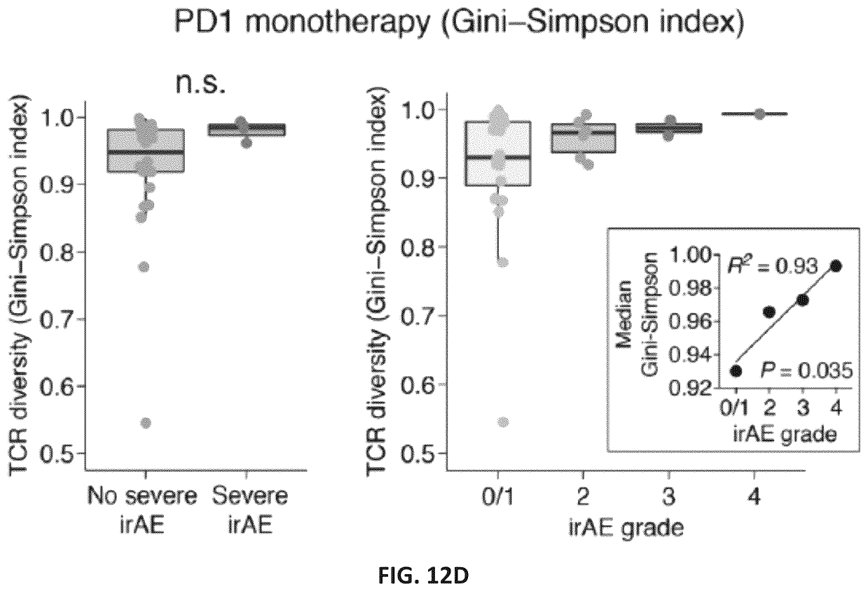

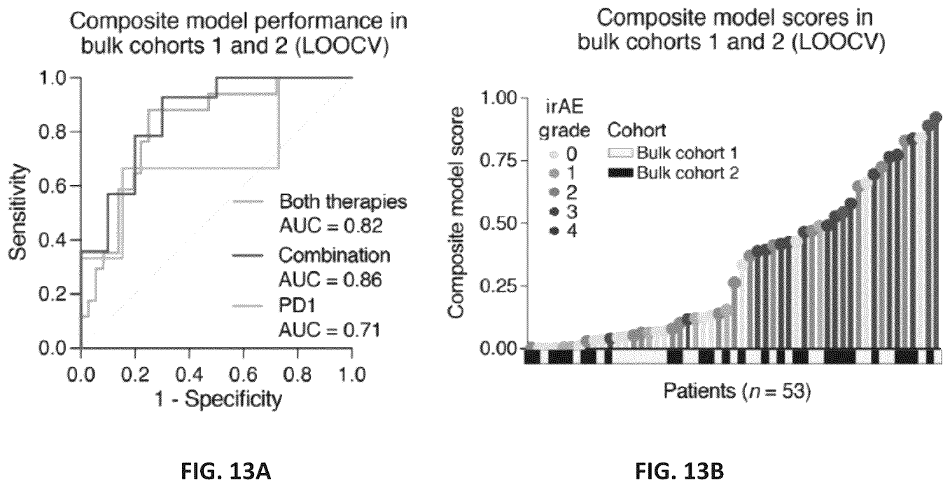

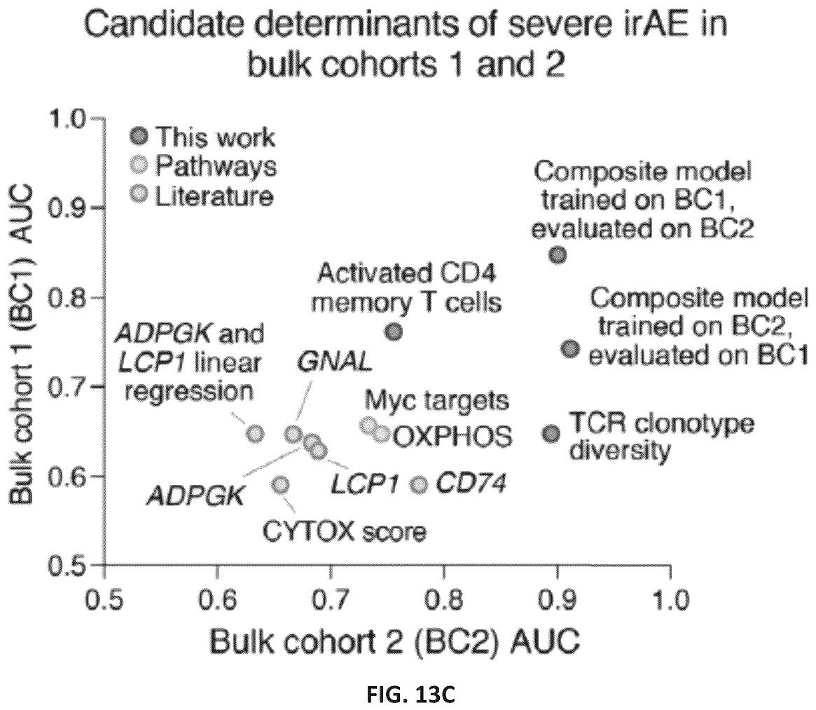

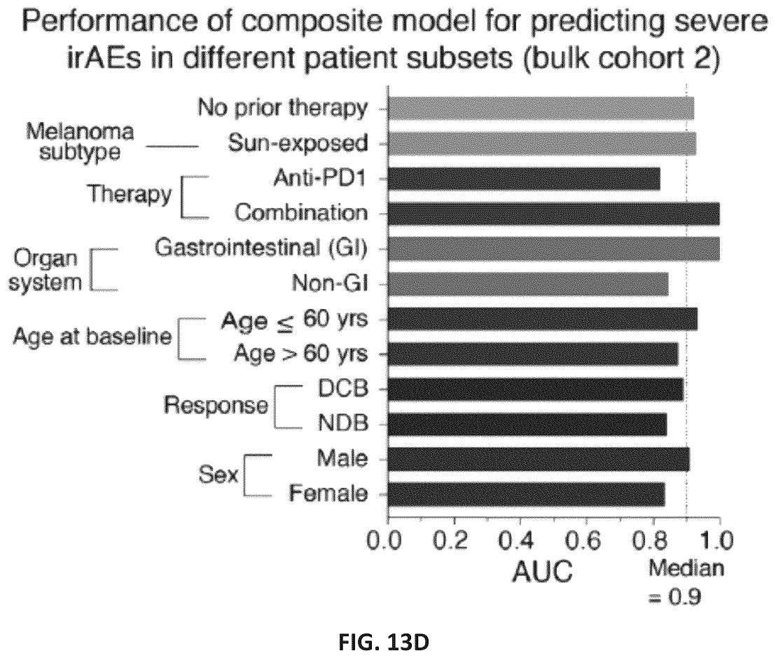

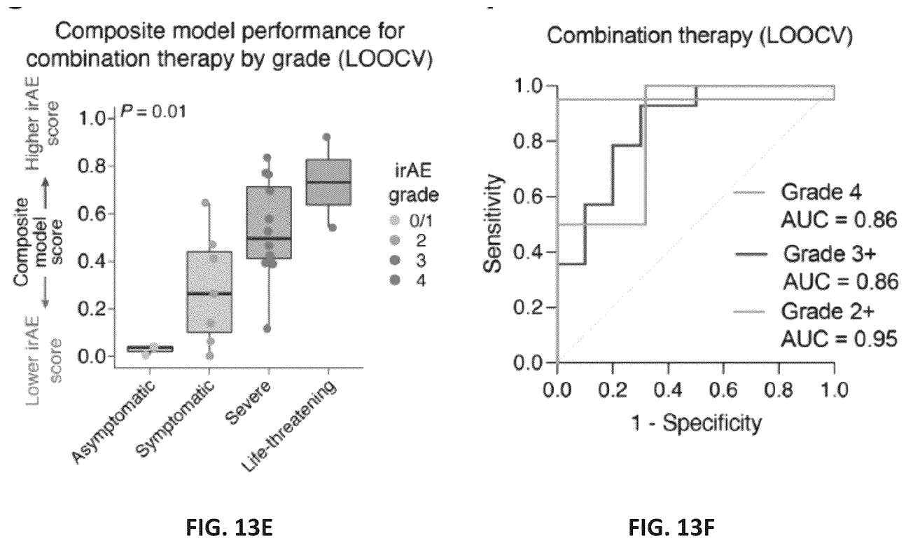

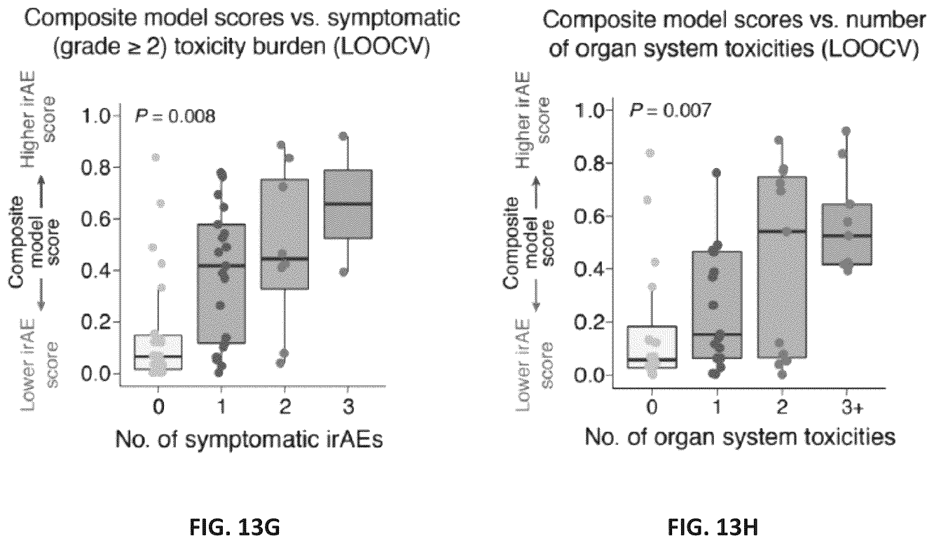

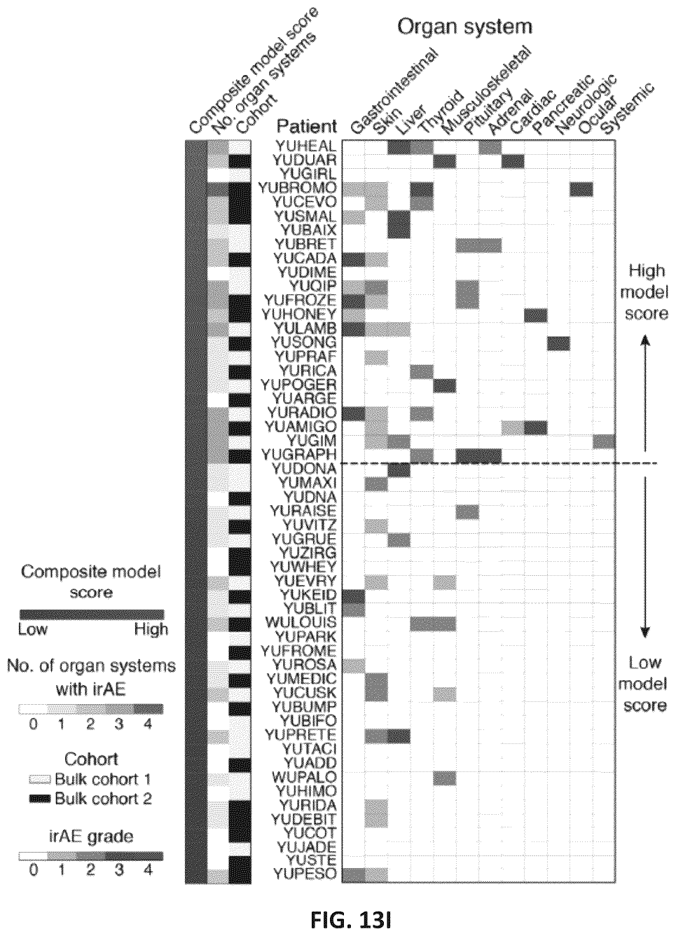

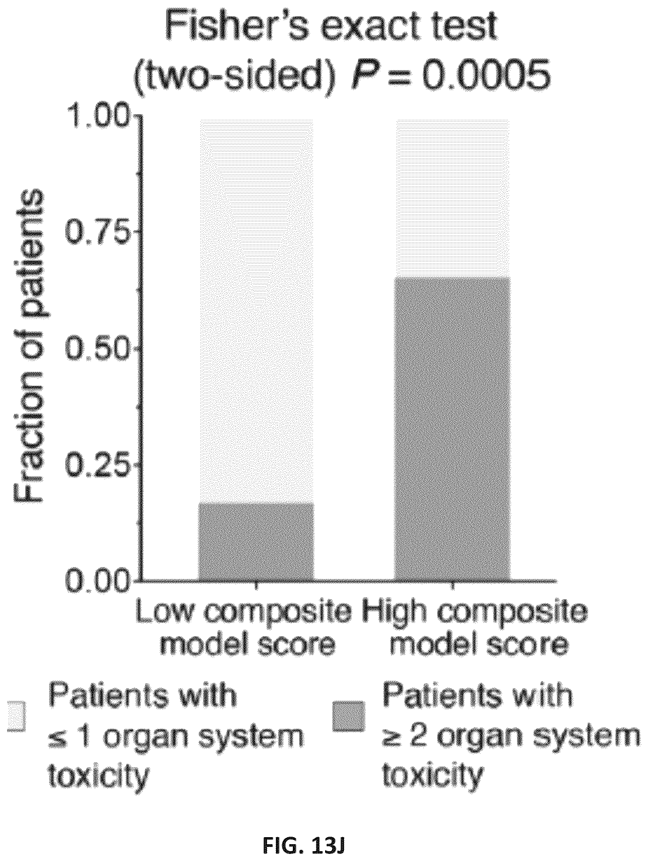

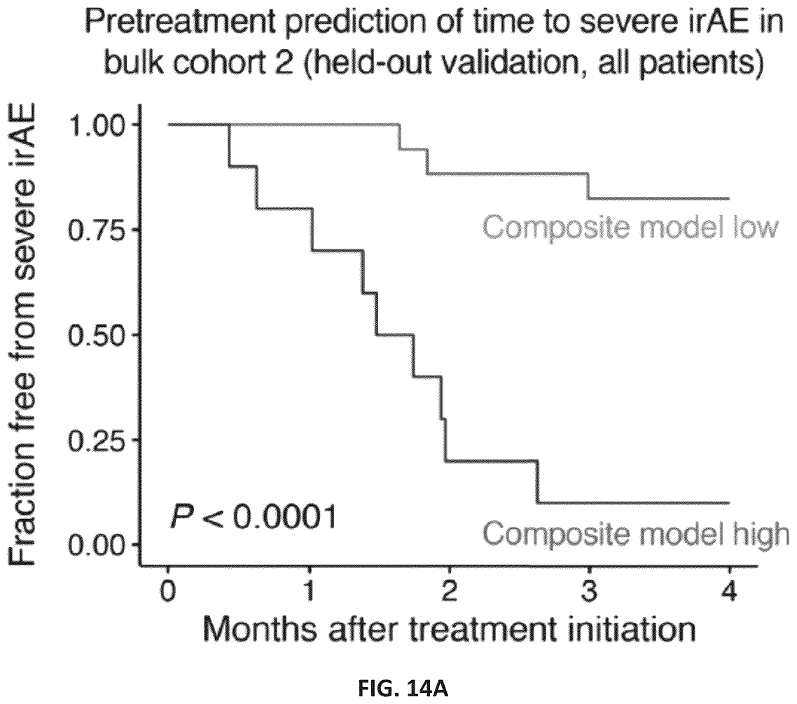

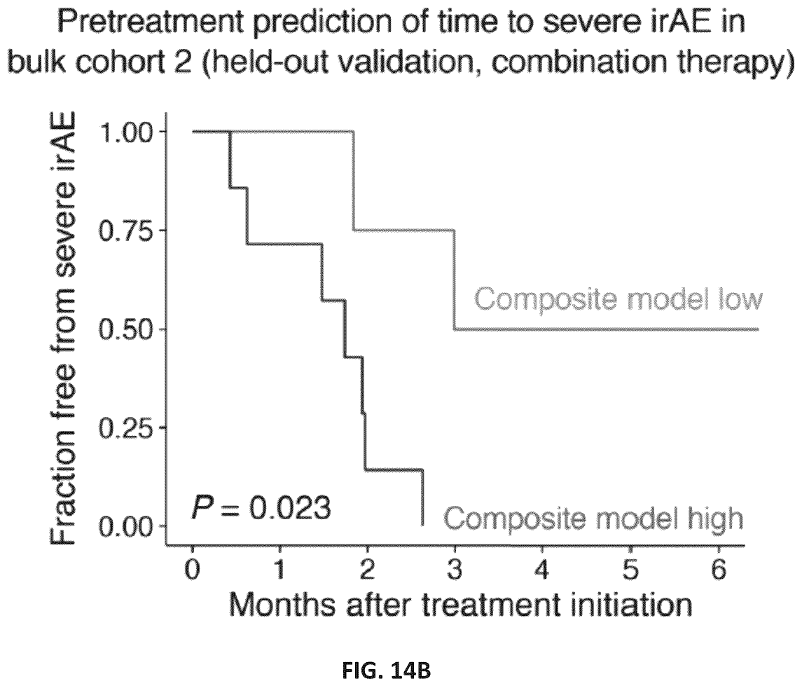

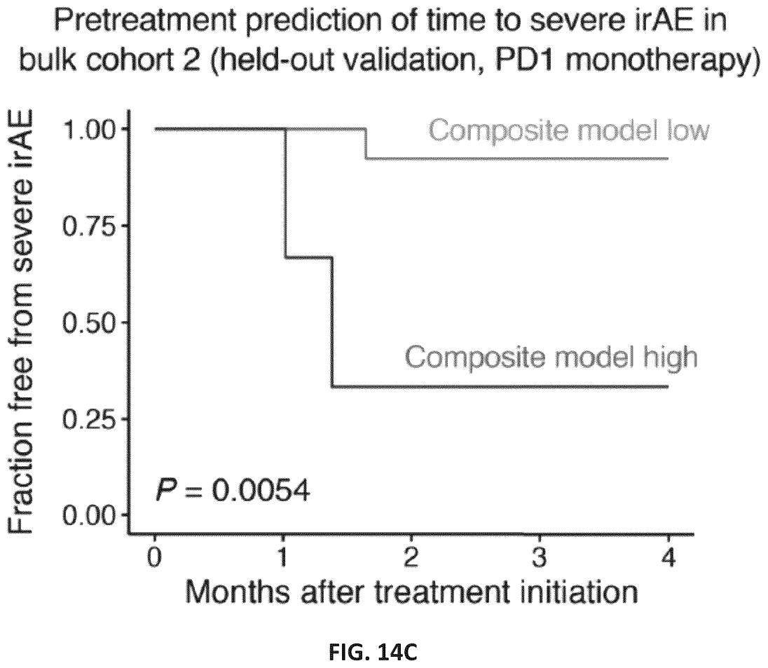

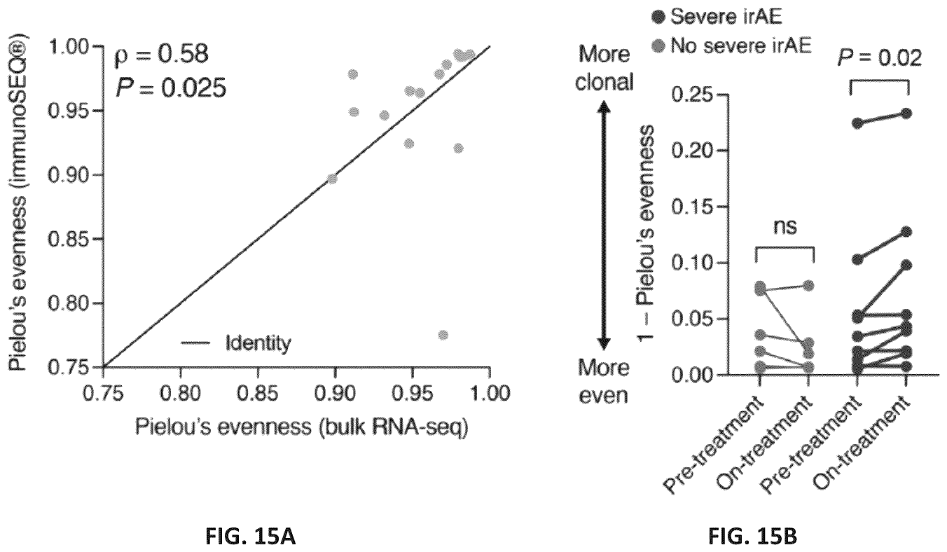

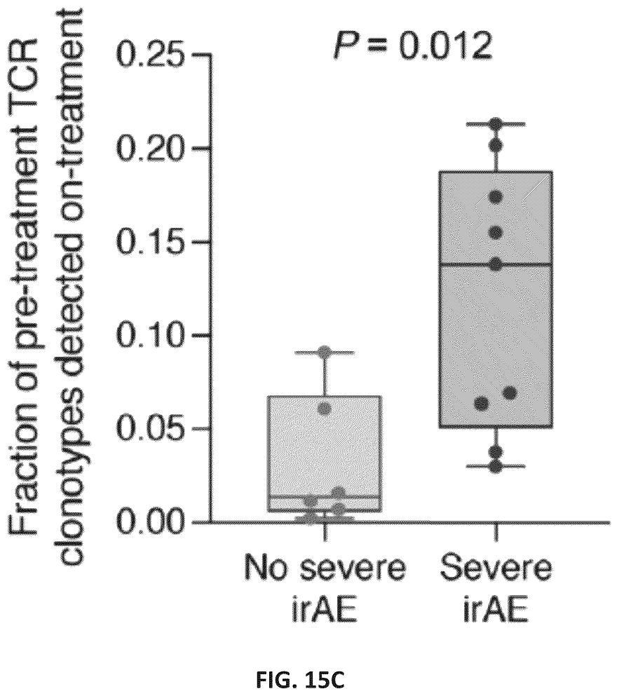

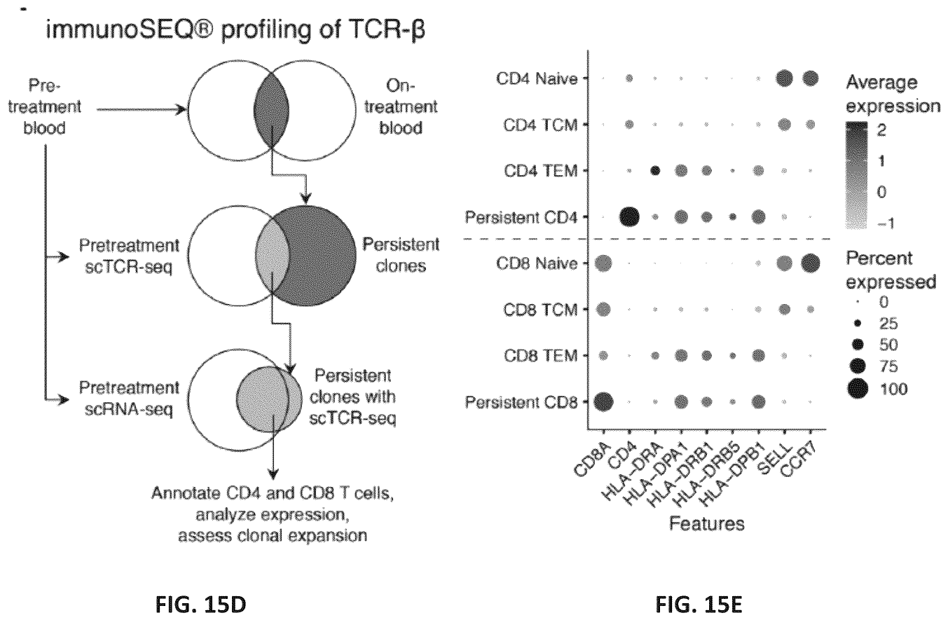

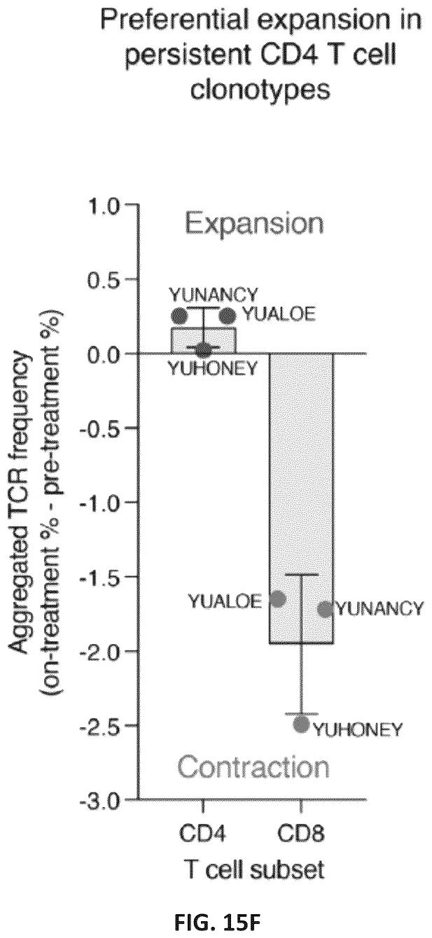

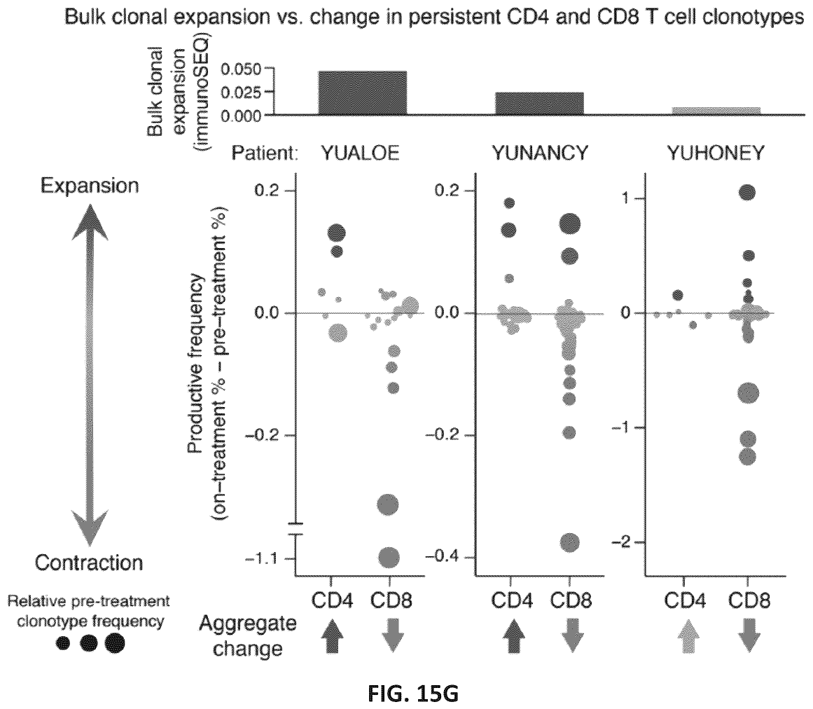

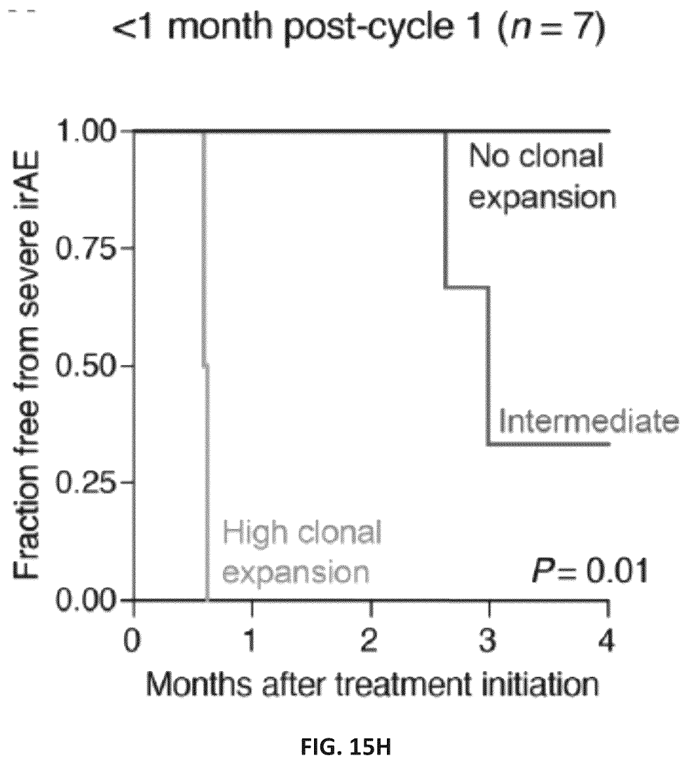

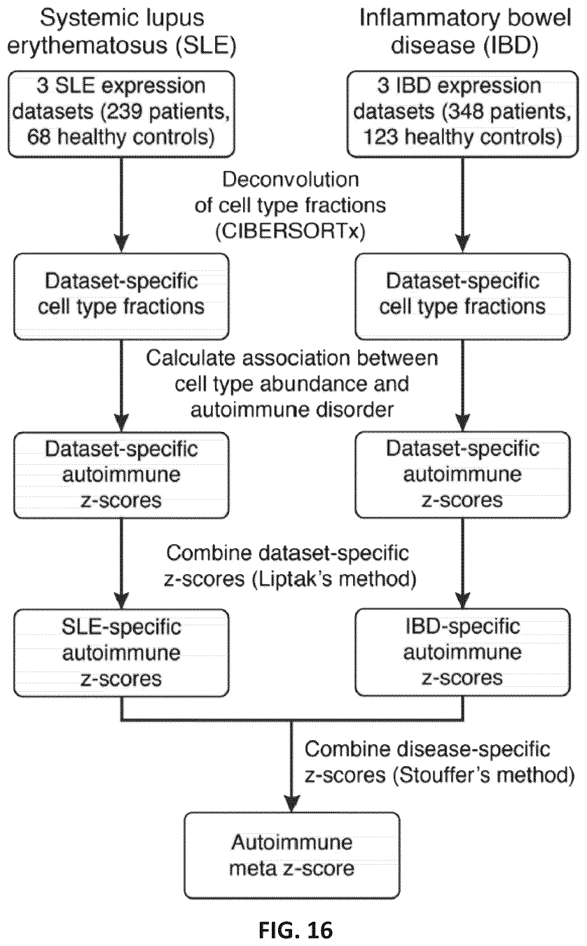

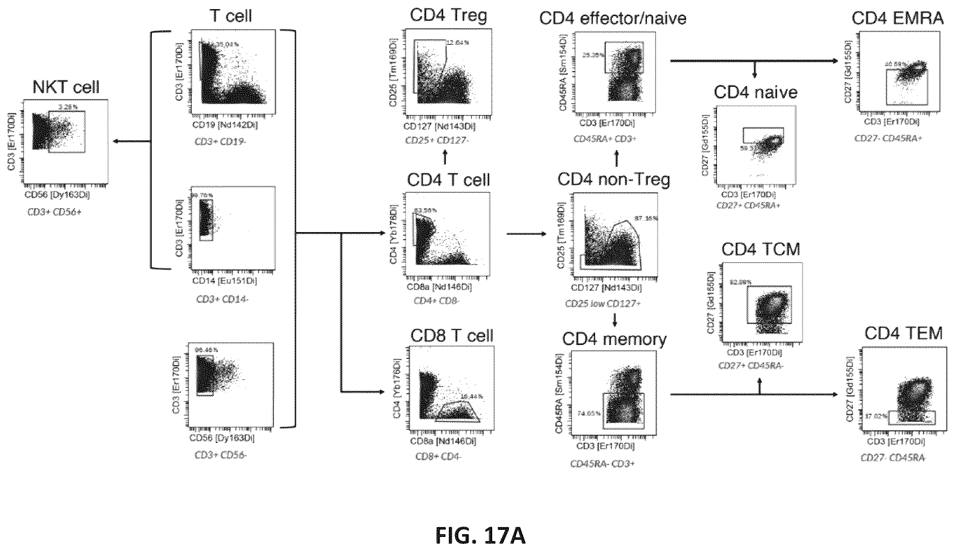

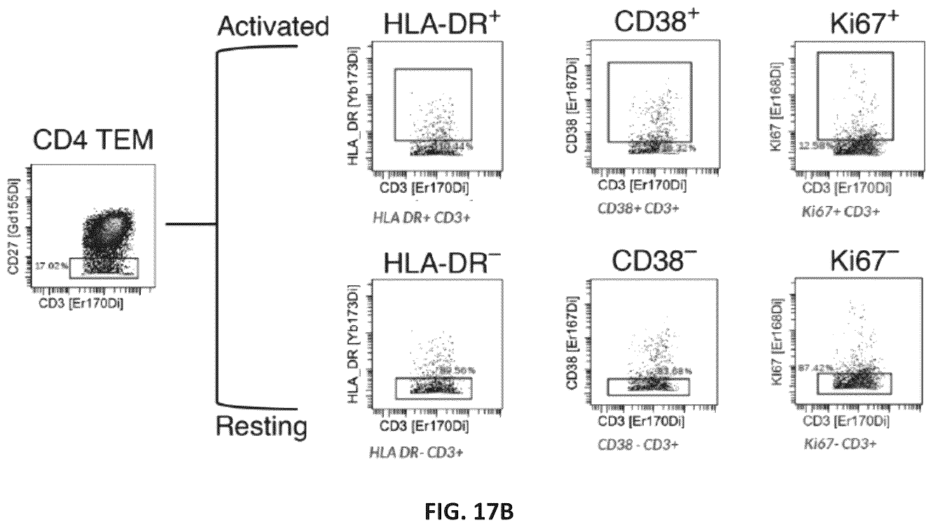

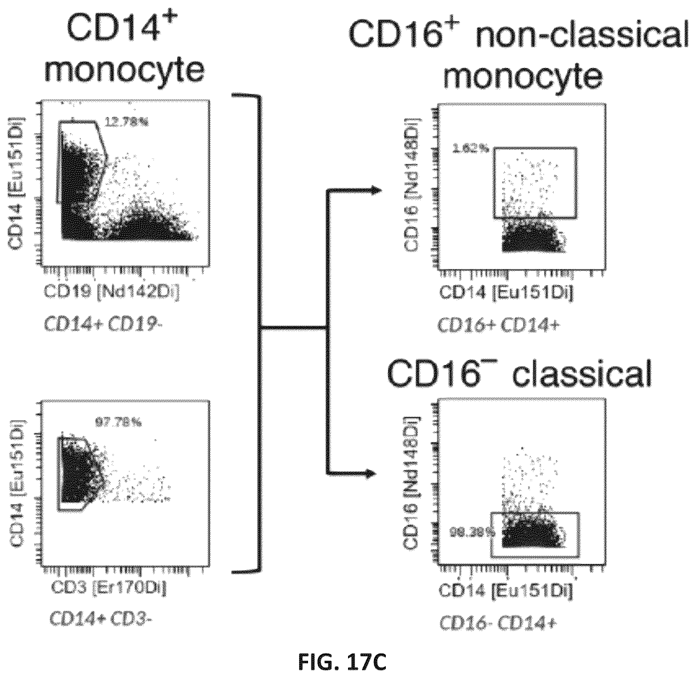

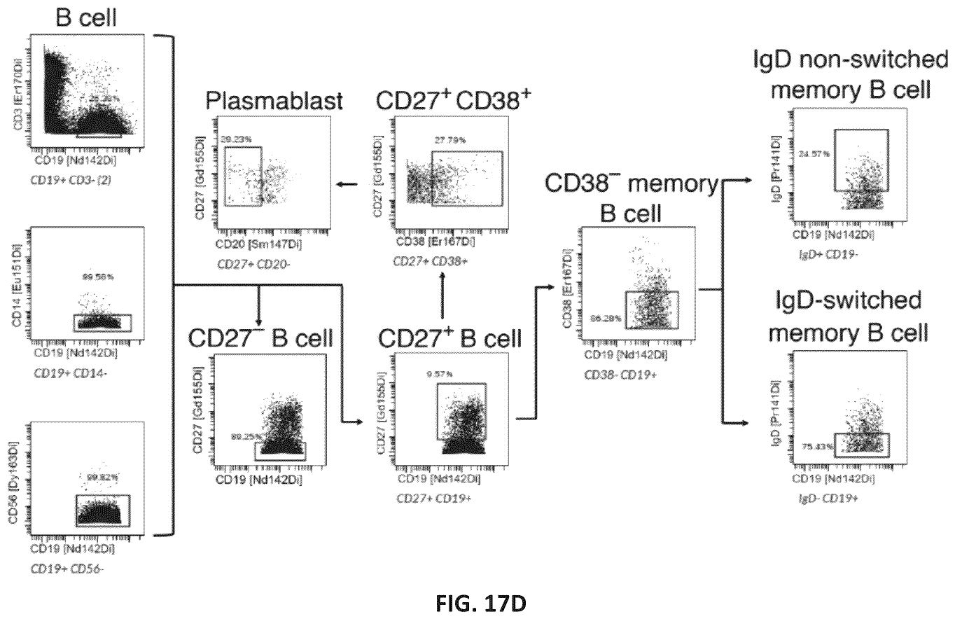

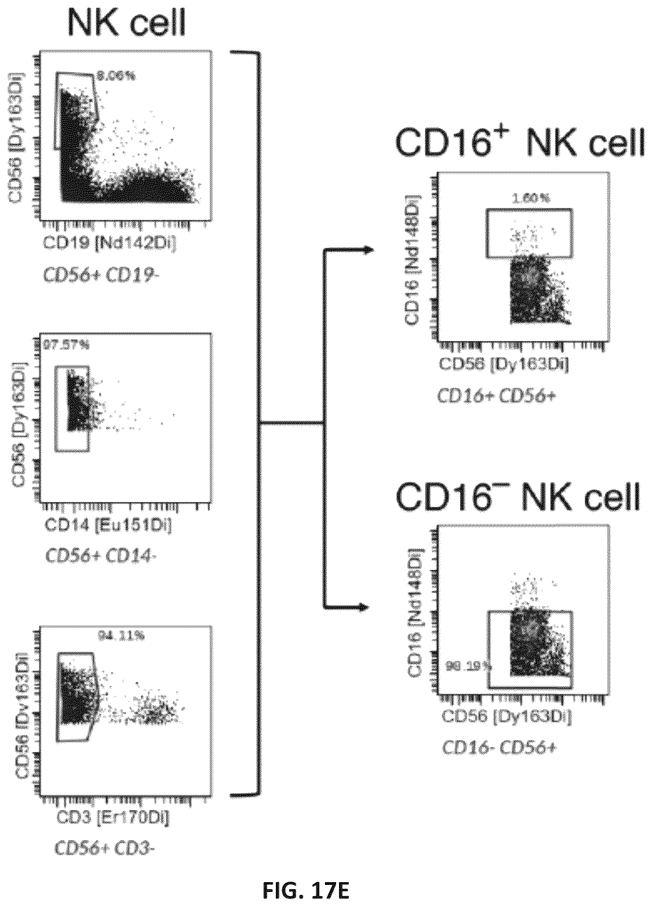

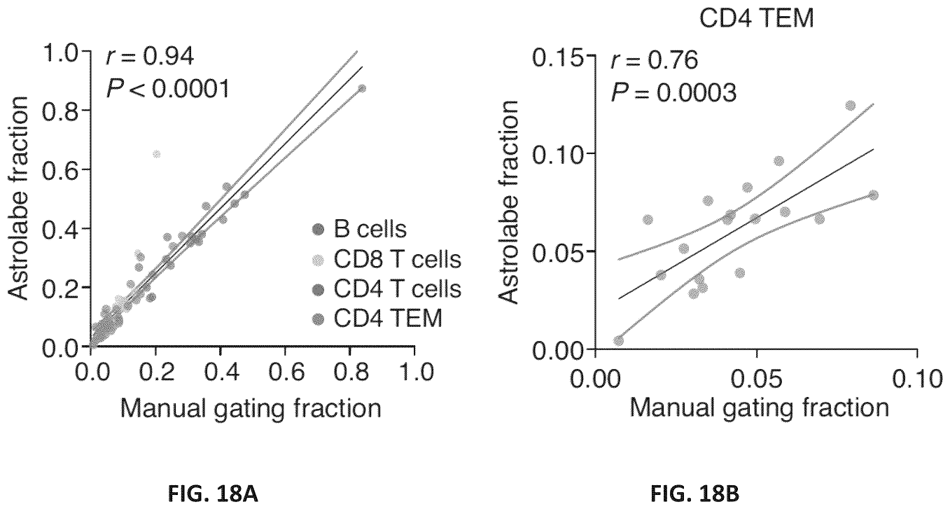

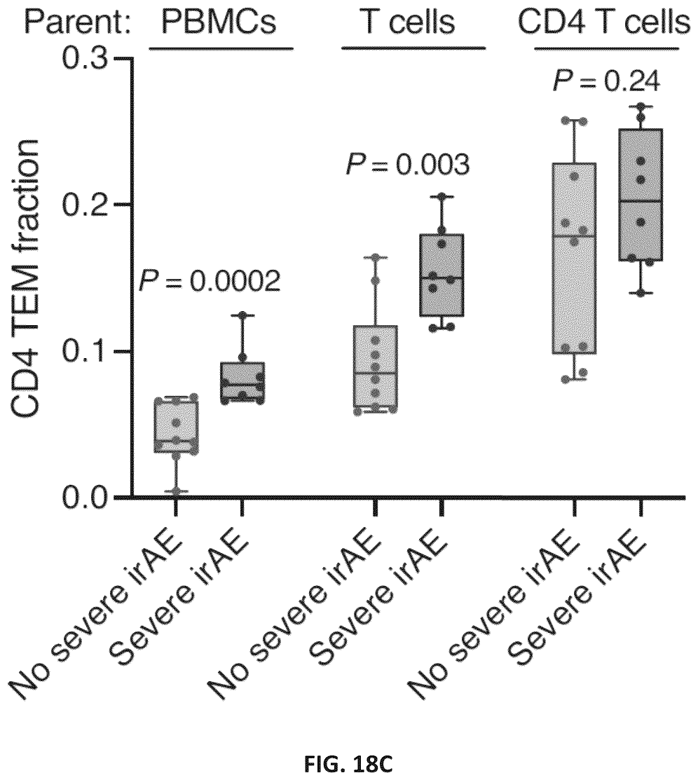

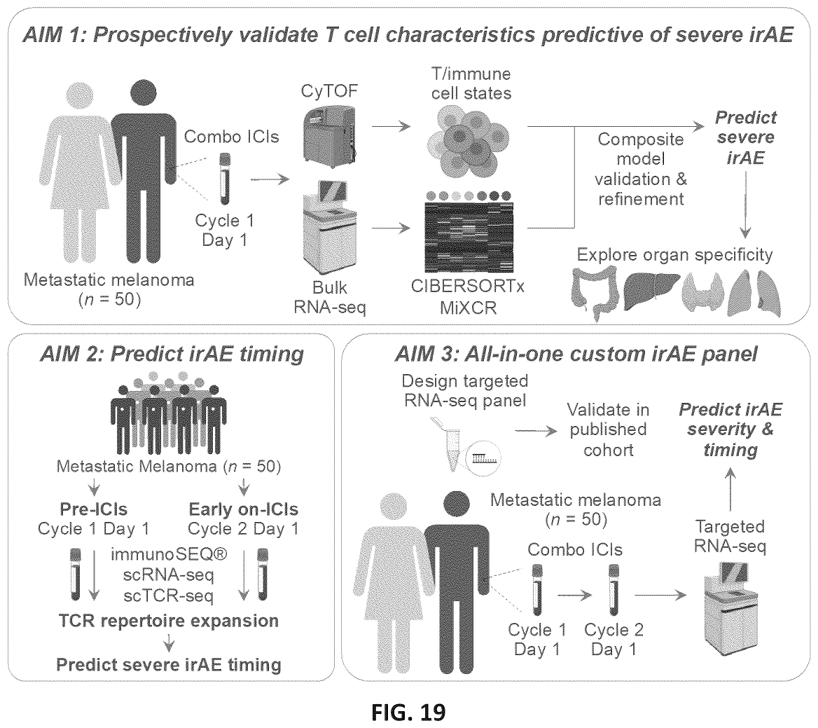

### DETAILED DESCRIPTION OF THE INVENTION

Severe immune-related adverse events (irAEs) occur in ˜60% of melanoma patients treated with combination immune checkpoint inhibitors (ICIs) and cause treatment-related morbidity and mortality. However, there is no reliable way to predict the development or timing of severe irAEs.

Pre-treatment and on-treatment analysis of cellular states and T cell receptors predict immunotherapy toxicity onset and timing. We specifically combine the abundance of activated CD4 T effector memory cells and the diversity of the T cell receptor repertoire in peripheral blood to yield a composite biomarker predictive of immunotherapy toxicity. Clonal expansion from pre- to on-treatment predicts the timing of severe toxicity. A targeted RNA sequencing panel enables this analysis in a practical and cost-effective manner.

Immunotherapy toxicities (immune-related adverse events) can be severe, dangerous, life-threatening, and deadly. We have no way in practice to predict them reliably, early or pre-treatment. Doing so would facilitate toxicity anticipation, earlier intervention, and more personalized and precise administration of immunotherapy.

In various aspects, methods of predicting immunotherapy toxicity in patients are disclosed. The disclosed methods are based on the discovery that two factors derived from the analysis of peripheral blood samples comprising activated CD4 memory T cell abundance levels and bulk TCR diversity strongly correlate with severe immunity-related adverse event (irAE) development. In various aspects, a liquid biopsy method of predicting immunotherapy toxicity in patients is disclosed that includes obtaining a peripheral blood sample from a subject prior to receiving an immunotherapy treatment. In various aspects, the method further includes quantifying an abundance of activated CD4 memory T cells and a diversity of T cell receptors (TCR) within the peripheral blood sample. In some aspects, the method further includes determining a model index predictive of the likelihood of the patient developing severe irAR, in which the model index comprises a combination of the abundance of activated CD4 memory T cells and a diversity of T cell receptors (TCR). The method further includes classifying the patient as likely to develop a severe irAR if the value of the model index exceeds a threshold value. In some aspects, the method further comprises predicting the severity of the irAR based on the value of the model index, wherein a higher value of the model index is predictive of a more severe irAR. The threshold for a higher value of the model index can be determined empirically or by reference to known clinical standards.

In various other aspects, methods of predicting immunotherapy toxicity in patients are disclosed that are based on the degree of TCR expansion, defined herein as the increase in the diversity of TCRs over an early period of immunotherapy relative to the pre-treatment diversity of TCRs. The methods include obtaining a first peripheral blood sample from the patient prior to initiation of an immunotherapy and obtaining a second peripheral blood sample early in the administration of an immunotherapy to the patient. The methods further include obtaining a first TCR diversity from the first peripheral blood sample and a second TCR diversity from the second peripheral blood sample. The methods further include subtracting the first TCR diversity from the second TCR diversity to obtain a degree of TCR expansion. In some aspects, the methods further include classifying the patient as likely to develop severe irAR if the degree of TCR expansion exceeds a threshold value. In some aspects, the methods further include predicting the time of onset of the severe irAR based on the degree of TCR expansion.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as many as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit the translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. All other sequences of the gene and its controlling regions may be numbered relative to this initiation site. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. Constructs can also include, but are not limited to, additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities, and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequences can be modulated with the help of art-known computer simulation programs that can produce a polypeptide with, for example, improved activity or altered regulation. Based on these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C. +16.6 (log₁₀[Na⁺])+0.41 (fraction G/C content)−0.63 (% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, |
| | Phe, |
| Leu (L) | Ile, Val, Met, Ala, |
| | Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, |
| | Ala |

Exemplary nucleic acids which may be introduced to a host cell include, for example, DNA sequences or genes from another species, or even genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33 (5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

As will be appreciated based upon the foregoing specification, the above-described aspects of the disclosure may be implemented using computer programming or engineering techniques including computer software, firmware, hardware, or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving media, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” and/or “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are examples only and are thus not limiting as to the types of memory usable for the storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Washington). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independent and separate from other components and processes described herein. Each component and process can also be used in combination with other assembly packages and processes. The present aspects may enhance the functionality and functioning of computers and/or computer systems.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### Examples

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

What is claimed is:

1. A method for predicting a likelihood of developing a severe immune-related adverse event (irAE) in a patient receiving an immunotherapy, the method comprising:
a. obtaining a peripheral blood sample from a subject prior to receiving an immunotherapy treatment;
b. quantifying an abundance of activated CD4 memory T cells and a diversity of T cell receptors (TCR) within the peripheral blood sample; and
c. classifying the patient as likely to develop a severe irAR if the abundance of activated CD4 memory T cells in combination with amounts of TCR exceeds a threshold value.

2. The method of claim 1, further comprising determining the threshold value by reference to known clinical standards.

3. The method of claim 1, wherein the abundance of activated CD4 memory T cells and the diversity of T cell receptors (TCR) are determined using at least one of: bulk RNA-sequencing (CIBERSORTx and MiXCR), mass cytometry by time of flight (CyTOF), immunoSEQ® TCR-β profiling, droplet-based scRNA-sequencing and scTCR-sequencing, and targeted RNA-sequencing using an RNA panel targeted to activated CD4 memory T cells.

4. A method for predicting a likelihood of developing a severe immune-related adverse event (irAE) in a patient receiving an immunotherapy, the method comprising:
a. obtaining a first peripheral blood sample from a subject prior to receiving an immunotherapy treatment and a second peripheral blood sample subsequent to the administration of the immunotherapy to the patient;
b. quantifying a first TCR diversity level from the first peripheral blood sample and a second TCR diversity level from the second peripheral blood sample;
c. obtaining a degree of TCR expansion by subtracting the first TCR diversity level from the second TCR diversity level;
d. classifying the patient as likely to develop severe irAR if the degree of TCR expansion exceeds a threshold value.

5. The method of claim 4, further comprising predicting a time of onset of the severe irAR based on the degree of TCR expansion, wherein a higher degree of TCR expansion is predictive of an earlier onset of severe irAR.

6. The method of claim 4, wherein the first and second TCR diversities are determined using at least one of: bulk RNA-sequencing (CIBERSORTx and MiXCR), mass cytometry by time of flight (CyTOF), immunoSEQ® TCR-β profiling, droplet-based scRNA-sequencing and scTCR-sequencing, and targeted RNA-sequencing using an RNA panel targeted to activated CD4 memory T cells.
